(19) United States
(12) Patent Application Publication
(10) Pub. No.: US 2004/0002526 A1
(43) Pub. Date: Jan. 1, 2004

(54) Phospholipase D inhibitors and uses thereof

(75) Inventors: J. Peter Klein, Vashon, WA (US); Anil M. Kumar, Puyallup, WA (US); Marc J. McKennon, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Cell Therapeutics, Inc., Seattle, WA

(21) Appl. No.: 10/405,059

(22) Filed: Apr. 1, 2003

Related U.S. Application Data
(60) Provisional application No. 60/369,719, filed on Apr. 3, 2002.

(51) Int. Cl. A61K 31/427; A61K 31/426

(52) U.S. Cl. 514/369

(57) ABSTRACT

The invention is directed to thiazolidinones and the use thereof to inhibit phospholipase D (PLD) activity. The invention further relates to methods of treating cancer and inflammatory diseases using thiazolidinones.

### CROSS REFERENCE TO RELATED APPLICATION

This application claims the benefit of U.S. Provisional Patent Application No. 60/369,719, filed Apr. 3, 2002, where this provisional application is incorporated herein by reference in its entirety.

### BACKGROUND OF THE INVENTION

1. Field of the Invention

The invention is in the field of organic and medicinal chemistry. In particular, the invention relates to thiazolidinones and the use thereof to inhibit phospholipase D (PLD) activity. The invention further relates to methods of treating cancer and inflammatory diseases using thiazolidinones.

2. Description of the Related Art

PLD catalyzes the hydrolysis of phosphatidylcholine (PC) to phosphatidic acid (PA) and choline. PA is the precursor molecule for certain phosphoglycerides, such as phosphatidylinositol, and diacylglycerols, which are necessary for the production of other phosphoglycerides, such as phosphatidylcholine, and for triacylglycerols, which are essential biological fuel molecules.

In addition to being a crucial precursor molecule in biosynthetic reactions, PA has been added to the list of intercellular lipid messenger molecules. PA is a key messenger in a common signaling pathway activated by proinflammatory mediators such as interleukin-1β, tumor necrosis factor a, platelet activating factor, and lipid A. Bursten et al., Am. J. Physiol. 262:C328 (1992); Bursten et al., J. Biol. Chem. 255:20732 (1991); Kester, J. Cell Physiol. 156:317 (1993). PA has been implicated in mitogenesis of several cell lines. English, Cell Signal 8:341 (1996). PLD activity and PA level have been found to be increased in either ras or fps transformed cell lines compared to the parental Rat2 fibroblast cell line. Martin et al., Oncogene 14:1571 (1997). Activation of Raf-1, an essential component of the mitogen-activated protein kinase (MAPK) signaling cascade, by extracellular signals is initiated by association with intracellular membranes. Recruitment of Raf-1 to membranes has been reported to be mediated by direct association with PA generated through PLD activation. Rizzo et al., J. Biol. Chem. 275:23911-8 (2000). PLD has also been shown to cooperate with the epidermal growth factor receptor to transform 3Y1 rat fibroblasts. Lu et al., Mol. Cell Biol. 20:462 (2000). As an enzyme that generates PA in cells, therefore, PLD may play a role in cancer, and may also mediate inflammatory responses to various proinflammatory agents. Compounds that would modulate PLD activity, and hence alter the PA level involved in cell activation, therefore may be of therapeutic interest in the area of oncology and inflammation.

A number of agents are known in the art for the modulation of PLD activity in basic research and as putative therapeutic agents to treat a variety of conditions associated with aberrant PLD activity. Some of these agents include α and β-synucleins (Jenco et al., Biochemistry 37:4901 (1998)); amphiphysins (Lee et al., J. Biol. Chem. 275:18751(2000)); suramin, suramin analog (NSC 79741) D-609, ET-18-CH₃, U-73,122 (Gratas and Powis, Anticancer Research 13:1239 (1993)); and the ketoepoxides (Pai et al., Anti-cancer Drug Design 9:363 (1994); Chu et al., Bioorg. Med. Chem. Lett. 4:1539 (1994)). However, thiazolidinones of the present invention have not been applied as agents in the treatment of conditions associated with aberrant PLD activity.

U.S. Pat. No. 5,216,002 discloses compounds, stated to be useful in the treatment of inflammatory bowel disease, of the general structure:

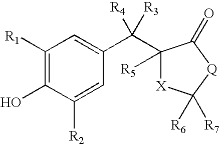

Published PCT Application WO 00/10573 discloses compounds of the general structure:

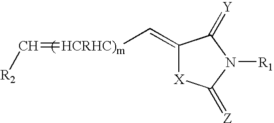

along with related compositions and methodology, stated to be useful for treating or preventing viral infections and associated diseases.

A variety of compounds also have been shown to be aldose reductase inhibitors in the context of diabetes. Ohishi et al., Chem. Pharm. Bull. 38:1911 (1990), 40:907 (1992); Fresneau et al., J. Med. Chem. 41:4706 (1998). Ono Pharmaceutical Company in Japan has marketed a compound, with the generic name “Epalrestat” ([5-(2-methyl-3-phenyl-allylidene)-4-oxo-2-thioxo-thiazolidin-3-yl]-acetic acid; CAS# 82159-09-9), for treating diabetic neuropathy.

Published reports further identify compounds that exhibit antimicrobial antibacterial, antiprotozoal, and antifungal activity, respectively. Mallick and Martin, J. Med. Chem. 14:528 (1971). For example, nitrodan (3-methyl-5-(4-nitrophenylazo)-2-thioxo-4-thiazolidinone) exhibits antimicrobial activity.

Yet other compounds have been shown to inhibit interaction between the BH3 domain and BCl-x_{L} in the context of studying the role of the BH3 domain in mediating pro-apoptotic and anti-apoptotic activities. Degterev et al., Nature Cell Biol. 3:173 (2001). Lastly, certain radiolabeled compounds have been synthesized as high-affinity, nonsteroidal androgen receptor ligands, for use as imaging agents for positron emission tomography (PET) and single-photon emission computed tomography. Van Dort et al., J. Med. Chem. 43:3344 (2000).

### BRIEF SUMMARY OF THE INVENTION

The inventors have discovered that thiazolidinones of the present invention surprisingly modulate PLD activity at low micromolar concentrations and, hence, alter the PA level involved in cell activation. Accordingly, the compounds are of therapeutic interest in the area of oncology and inflammation. In addition, the thiazolidinones of the present invention are readily synthesized, by means of standard organic chemistry transformation, using readily available starting materials.

The invention relates to a method for inhibiting PLD (phospholipase D) comprising contacting PLD with an effective amount of a thiazolidinone or a pharmaceutically acceptable salt, composition and prodrug thereof, thereby inhibiting PLD.

The invention further relates to inhibiting cell proliferation by contacting a cell with an effective amount of a thiazolidinone, or a pharmaceutically acceptable salt, composition and prodrug thereof, thereby inhibiting the proliferation of the cell.

The invention relates to a method for treating cancer, comprising administering to an animal in need thereof, an effective amount of a thiazolidinone, or a pharmaceutically acceptable salt, composition and prodrug thereof, wherein the cancer is treated.

The invention relates to a method for treating inflammation, comprising administering to an animal in need thereof, an effective amount of a thiazolidinone, or a pharmaceutically acceptable salt, composition and prodrug thereof, wherein the inflammation is treated.

### DETAILED DESCRIPTION OF THE INVENTION

Prior to setting forth the invention, it may be helpful to an understanding thereof to set forth definitions of certain terms to be used hereinafter.

In the present description, the term “alkyl” refers to straight- or branched-chain hydrocarbons having from 1 to 10 carbon atoms and more preferably 1 to 8 carbon atoms which include, by way of example, methyl, ethyl, n-propyl, i-propyl, n-butyl, t-butyl and the like. The alkyl group may be substituted or unsubstituted. When substituted, the substituted group(s) is preferably one or more independently selected from alkyl, cycloalkyl, heteroalicyclic, aryl, heteroaryl, haloalkyl, halo, hydroxy, alkoxy, mercapto, cyano, sulfonamidyl, aminosulfonyl, acyl, acyloxy, and substituted amino.

“Alkenyl” includes monovalent hydrocarbon radicals having straight, cyclic, or branched moieties, and combinations thereof which comprise at least one carbon-carbon double bond. The alkenyl group may be substituted or unsubstituted. When substituted, the substituted group(s) is preferably one or more independently selected from alkyl, acyl, cycloalkyl, heteroalicyclic, aryl, haloalkyl, alkoxy and substituted amino.

“Alkoxy” refers to the group “alkyl-O-” which includes, by way of example, methoxy, ethoxy, n-propoxy, i-propoxy, n-butoxy, t-butoxy and the like.

“Substituted amino” denotes the group —NRR, wherein each R group is independently selected from hydrogen, acyl, alkyl, cycloalkyl, aryl, or the R groups can be joined together with the nitrogen to form a heterocyclic ring (e.g., piperidine, piperazine, or a morpholine ring).

“Aryl” refers to an unsaturated aromatic carbocyclic group of 6 to 14 carbon atoms having a single ring (e.g., phenyl) or multiple condensed rings (e.g., naphthyl or anthryl). The aryl group may be unsubstituted or substituted; in the latter case, the substituent or substituents preferably are selected independently from alkyl, aryl, haloalkyl, halo, hydroxy, alkoxy, mercapto, cyano, sulfonamidyl, aminosulfonyl, acyl, acyloxy, nitro, and substituted amino.

“Heteroaryl” is a monocyclic or fused ring (i.e., rings which share an adjacent pair of atoms) group having in the ring(s) one or more atoms selected preferably from nitrogen, oxygen and sulfur and, in addition, having a completely conjugated π-electron system. Exemplary heteroaryl groups are pyrrole, furan, thiophene, imidazole, oxazole, thiazole, pyrazole, pyridine, pyrimidine, quinoline, isoquinoline, purine and carbazole. The heteroaryl group may be substituted or unsubstituted. When substituted, the substituted group(s) is preferably one or more independently selected from alkyl, aryl, haloalkyl, halo, hydroxy, alkoxy, mercapto, cyano, sulfonamidyl, aminosulfonyl, acyl, acyloxy, nitro and substituted amino.

“Cycloalkyl” encompasses cyclic alkyl groups that contain between 3 and 8 carbon atoms and have a single cyclic ring, illustrated by cyclopropyl, cyclobutyl, cyclopentyl, and cyclooctyl. The cycloalkyl ring may be substituted or unsubstituted.

Again, a substituted cycloalkyl ring carries one or more substituent groups, independently selected preferably from alkyl, aryl, haloalkyl, halo, hydroxy, alkoxy, mercapto, cyano, sulfonamidyl, aminosulfonyl, acyl, acyloxy, vitro, and substituted amino.

“Heteroalicyclic” refers to a monocyclic or fused ring group having in the ring(s) one or more atoms selected preferably from nitrogen, oxygen and sulfur. The rings may also have one or more double bonds. However, the rings do not have a completely conjugated i-electron system. The heteroalicyclic ring may be substituted or unsubstituted. When substituted, the substituted group(s) preferably are selected independently from alkyl, aryl, haloalkyl, halo, hydroxy, alkoxy, mercapto, cyano, sulfonamidyl, aminosulfonyl, acyl, acyloxy, vitro, and substituted amino.

“Halogen” or “halo” refers to fluoro, chloro, bromo, iodo.

“Acyl” group refers to the —C(O)—R″ group, where R″ is selected preferably from hydrogen, hydroxy, alkyl, haloalkyl, cycloalkyl, aryl optionally substituted with one or more alkyl, haloalkyl, alkoxy, halo and substituted amino groups, heteroaryl (bonded through a ring carbon) optionally substituted with one or more alkyl, haloalkyl, alkoxy, halo and substituted amino groups and heteroalicyclic (bonded through a ring carbon) optionally substituted with one or more alkyl, haloalkyl, alkoxy, halo and substituted amino groups. Preferred acyl groups are carboxy groups, e.g., acids and esters.

The phrase “pharmaceutically acceptable salt” refers to those salts that retain the biological effectiveness and properties of the particular compound. Pharmaceutically acceptable salts are often useful because they may have improved stability and/or solubility in pharmaceutical compositions over the free base form of the compound. A pharmaceutically acceptable salt may be obtained by reaction of a free base with an inorganic acid such as hydrochloric acid, hydrobromic acid, nitric acid, phosphoric acid, sulfuric acid, and perchloric acid and the like, or with an organic acid such as acetic acid, oxalic acid, malic acid, maleic acid, methanesulfonic acid, ethanesulfonic acid, p-toluenesulfonic acid, salicylic acid, tartaric acid, citric acid, succinic acid or malonic acid, and the like. A pharmaceutically acceptable salt may also be obtained by reaction of a free acid with a base such as sodium, potassium or lithium hydroxide, bicarbonate or carbonate, and the like.

“Prodrug” herein refers to a compound that is converted into the parent compound in vivo. Prodrugs often are useful because, in some situations, they may be easier to administer than the parent compound. They may, for instance, be bioavailable by oral administration whereas the parent compound is not. The prodrug may also have improved solubility in pharmaceutical compositions over the parent compound. An example of a prodrug would be a compound of the embodiments of the present invention which is administered as an ester (the “prodrug”) to facilitate transmittal across a cell membrane where water solubility is detrimental to mobility, but then is metabolically hydrolyzed to the carboxylic acid, the active entity, once inside the cell where water solubility is beneficial. Such a compound is generally inactive (or less active) until converted to the active form.

I. The Compounds

The present invention relates to a method for inhibiting PLD comprising contacting PLD with an effective amount of a thiazolidinone or a pharmaceutically acceptable salt, composition and prodrug thereof, thereby inhibiting PLD. The thiazolidinones contemplated by the preferred embodiments of the present invention fall under three categories: monocyclic, bicyclic and tricyclic thiazolidinones. Preferred monocyclic thiazolidinones are those of the Formula I:

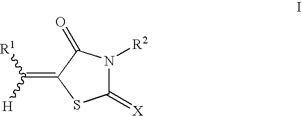

wherein R¹ is hydrogen, alkenyl, aryl or heteroaryl; R² is hydrogen, alkyl, cycloalkyl, heteroalicyclic, aryl, arylalkyl or arylalkenyl; X is O or S; or pharmaceutically acceptable salts, compositions and prodrugs thereof. Preferred R¹ and R² are where R¹ is selected from H; alkyl or aryl substituted alkyl; alkyl or aryl substituted alkenyl; unsubstituted, halo, hydroxy, alkoxy, amino, alkylamino, dialkylamino, alkoxycarbonyl, aminocarbonyl, alkylmercapto or arylmercapto substituted aryl; halo, hydroxy, alkoxy, methylenedioxy, amino, alkylamino, dialkylamino, cyano, carboxy, alkoxycarbonyl, aminocarbonyl, sulfonamidyl or aminosulfonyl substituted aryl substituted furanyl, pyrrolyl or thiophenyl; halo, hydroxy, alkoxy, methylenedioxy, amino, alkylamino, dialkylamino, cyano, carboxy, alkoxycarbonyl, aminocarbonyl, aminosulfonyl or alkyl substituted aryl substituted alkyl or alkenyl; indolyl; pyridinyl; and R² is selected from H; unsubstituted or morpholino, halo, hydroxy, alkoxy, methylenedioxy, amino, alkylamino, dialkylamino, cyano, carboxy, alkoxycarbonyl, aminocarbonyl, alkylmercapto or arylmercapto substituted aryl; halo, hydroxy, alkoxy, methylenedioxy, amino, alkylamino, dialkylamino, cyano, carboxy, alkoxycarbonyl, aminocarbonyl, sulfonamidyl or aminosulfonyl pyridinyl, thiophenyl, furanyl, or pyrrolyl substituted cyclic or acyclic alkyl or alkenyl. Examples of particularly preferred R¹ substituents are halo substituted phenyl, aryl substituted thiophenyl, and aryl substituted furanyl (such as furan substituted with benzoic acid). Examples of particularly preferred R² substituents are halo substituted aryl, carboxy substituted aryl, and carboxy substituted acyclic alkyl (such as acetic acid, propionic acid, and butyric acid) and cyclic alkyl.

Preferred monocyclic thiazolidinones of the Formula I of the preferred embodiments of the present invention are shown in Table 1.

TABLE 1

| | | IC₅₀ | |
|---|---|---|---|
| | Compound | (μM) | Compound Name |
| 1 | 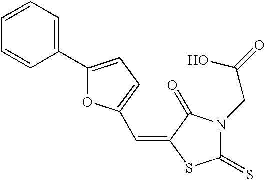 | 4 | [4-oxo-5-(5-phenyl-furan-2- ylmethylene)-2-thioxo- thiazolidin-3-yl]-acetic acid |
| 2 | 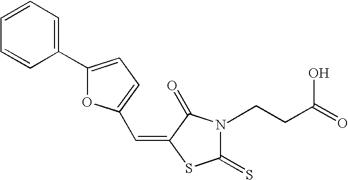 | 1.5 | 3-[4-oxo-5-(5-phenyl-furan-2- ylmethylene)-2-thioxo- thiazolidin-3-yl]-acetic acid |
| 3 | 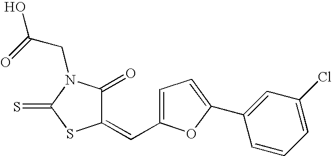 | 2 | {5-[5-(3-chloro-phenyl)-furan-2- ylmethylene]-4-oxo-2-thioxo- thiazolidin-3-yl}-acetic acid |
| 4 | 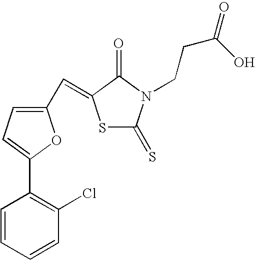 | 0.8 | 3-{5-[5-(2-chloro-phenyl)-furan- 2-ylmethylene]-4-oxo-2-thioxo- thiazolidin-3-yl}-propionic acid |
| 5 | 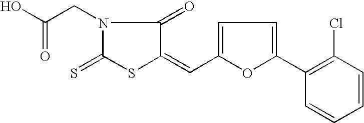 | 1.8 | {5-[5-(2-chloro-phenyl)-furan-2- ylmethylene]-4-oxo-2-thioxo- thiazolidin-3-yl}-acetic acid |
| 6 | 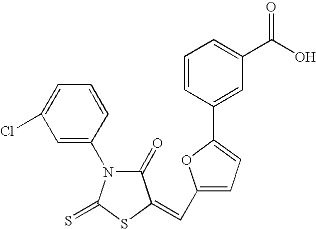 | 0.4 | 3-{5-[3-(3-chloro-phenyl)-4-oxo- 2-thioxo-thiazolidin-5- ylidenemethyl]-furan-2-yl}- benzoic acid |
| 7 | 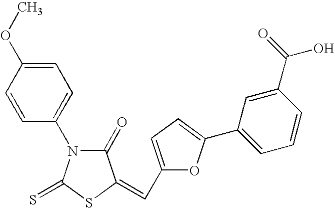 | 1.3 | 3-{5-[3-(4-methoxy-phenyl)-4- oxo-2-thioxo-thiazolidin-5- ylidenemethyl]-furan-2-yl}- benzoic acid |
| 8 | 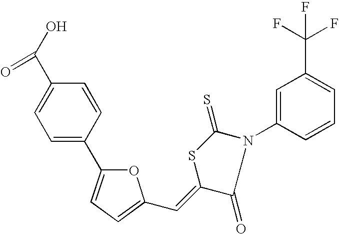 | 1.4 | 4-{5-[4-oxo-2-thioxo-3-(3- trifluoromethyl-phenyl)- thiazolidin-5-ylidenemethyl]- furan-2-yl}-benzoic acid |
| 9 | 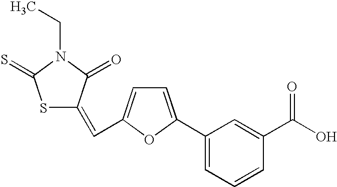 | 0.6 | 3-[5-(3-ethyl-4-oxo-2-thioxo- thiazolidin-5-ylidenemethyl)- furan-2-yl]-benzoic acid |
| 10 | 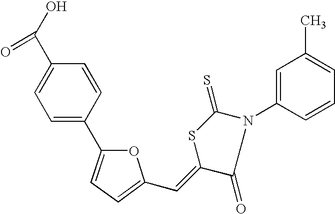 | 0.6 | 4-[5-(4-oxo-2-thioxo-3-m-tolyl- thiazolidin-5-ylidenemethyl)- furan-2-yl]-benzoic acid |
| 11 | 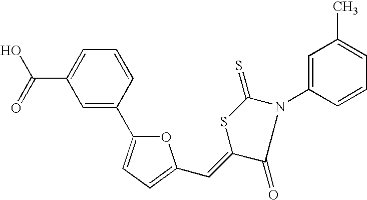 | <0.4 | 3-[5-(4-oxo-2-thioxo-3-m-tolyl- thiazolidin-5-ylidenemethyl)- furan-2-yl]-benzoic acid |
| 12 | 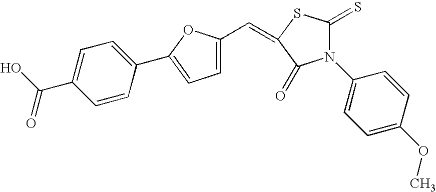 | <0.4 | 4-{5[3-(4-methoxy-phenyl)-4- oxo-2-thioxo-thiazolidin-5- ylidenemethyl]-furan-2-yl}- benzoic acid |
| 13 | 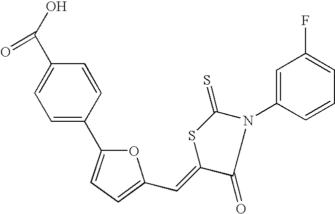 | 1.7 | 4-{5-[3-(3-fluoro-phenyl)-4-oxo- 2-thioxo-thiazolidin-5- ylidenemethyl]-furan-2-yl}- benzoic acid |
| 14 | 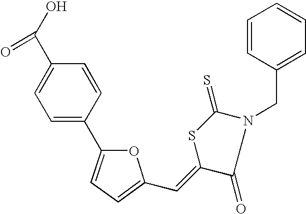 | <0.4 | 4-[5-(3-benzyl-4-oxo-2-thioxo- thiazolidin-5-ylidenemethyl)- furan-2-yl]-benzoic acid |
| 15 | 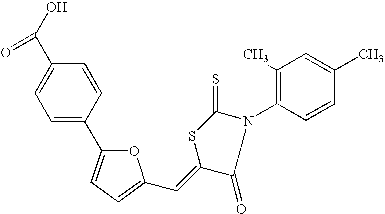 | <0.4 | 4-{5-[3-(2,4-dimethyl-phenyl)-4- oxo-2-thioxo-thiazolidin-5- ylidenemethyl]-furan-2-yl}- benzoic acid |
| 16 | 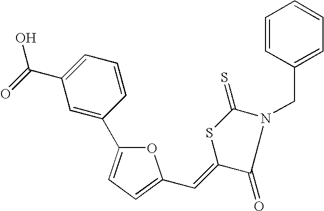 | <0.9 | 3-[5-(3-benzyl-4-oxo-2-thioxo- thiazolidin-5-ylidenemethyl)- furan-2-yl]-benzoic acid |
| 17 | 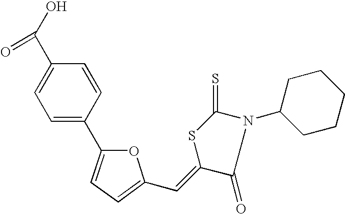 | 0.5 | 4-[5-(3-cyclohexyl-4-oxo-2- thioxo-thiazolidin-5- ylidenemethyl)-furan-2-yl]- benzoic acid |
| 18 | 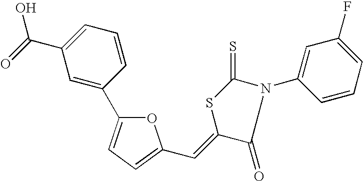 | <0.4 | 3-{5-[3 -(3-fluoro-phenyl)-4-oxo- 2-thioxo-thiazolidin-5- ylidenemethyl]-furan-2-yl}- benzoic acid |
| 19 | 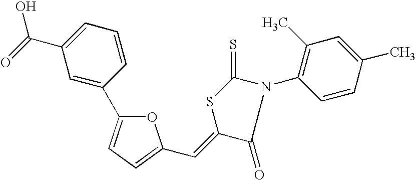 | <0.4 | 3-{5-[3-(2,4-dimethyl-phenyl)-4- oxo-2-thioxo-thiazolidin-5- ylidenemethyl]-furan-2-yl}- benzoic acid |
| 20 | 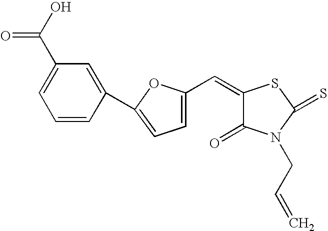 | 0.5 | 3-[5-(3-allyl-4-oxo-2-thioxo- thiazolidin-5-ylidenemethyl)- furan-2-yl]-benzoic acid |
| 21 | 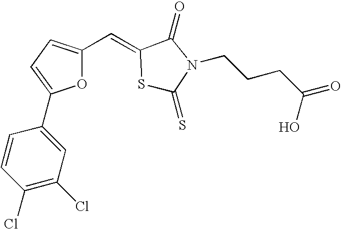 | 0.4 | 4-{5-[5-(3,4-dichloro-phenyl)- furan-2-ylidenemethyl]-4-oxo-2- thioxo-thiazolidin-3-yl}-butyric acid |
| 22 | 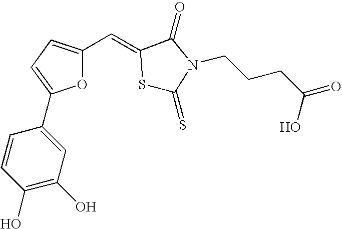 | 1.6 | 4-{5-[5-(3,4-dihydroxy-phenyl)- furan-2-ylidenemethyl]-4-oxo-2- thioxo-thiazolidin-3-yl}-butyric acid |
| 23 | 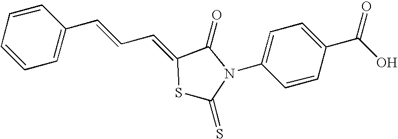 | 9 | 4-[4-oxo-5-(3-phenyl-allylidene)- 2-thioxo-thiazolidin-3-yl]- benzoic acid |
| 24 | 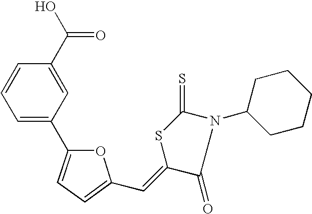 | <0.4 | 3-[5-(3-Cyclohexyl-4-oxo-2- thioxo-thiazolidin-5- ylidenemethyl)-furan-2-yl]- benzoic acid |
| 25 | 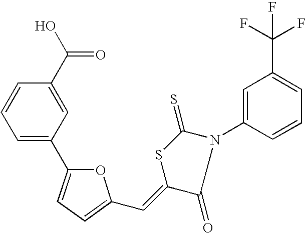 | <0.4 | 3-{5-[4-oxo-2-thioxo-3-(3- trifluoromethyl-phenyl)- thiazolidin-5-ylidenemethyl]- furan-2-yl}-benzoic acid |
| 26 | 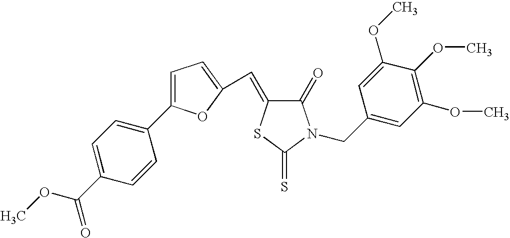 | 3 | 4-{5-[4-oxo-2-thioxo-3-(3,4,5- trimethoxy-benzyl)-thiazolidin- 5-ylidenemethyl]-furan-2-yl}- benzoic acid methyl ester |
| 27 | 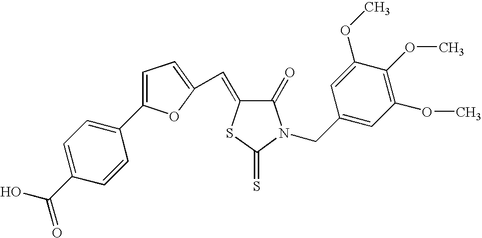 | 3 | 4-{5-[4-oxo-2-thioxo-3-(3,4,5- trimethoxy-benzyl)-thiazolidin- 5-ylidenemethyl]-furan-2-yl}- benzoic acid |
| 28 | 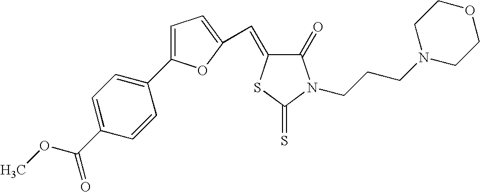 | 1.5 | 4-{5-[3-(3-morpholin-4-yl- propyl)-4-oxo-2-thioxo- thiazolidin-5-ylidenemethyl]- furan-2-yl}-benzoic acid methyl ester |
| 29 | 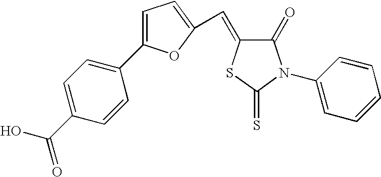 | 4 | 4-[5-(4-oxo-3-phenyl-2-thioxo- thiazolidin-5-ylidenemethyl)- furan-2-yl]-benzoic acid |
| 30 | 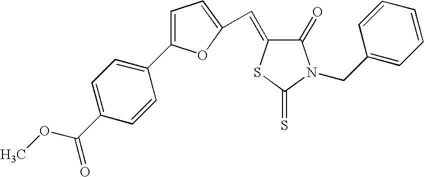 | 16 | 4-[5-(3-benzyl-4-oxo-2-thioxo- thiazolidin-5-ylidenemethyl)- furan-2-yl]-benzoic acid methyl ester |
| 31 | 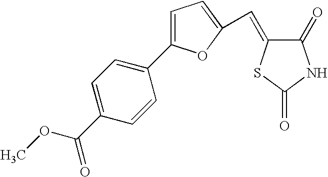 | less active | 4-[5-(2,4-dioxo-thiazolidin-5- ylidenemethyl)-furan-2-yl]- benzoic acid methyl ester |
| 32 | 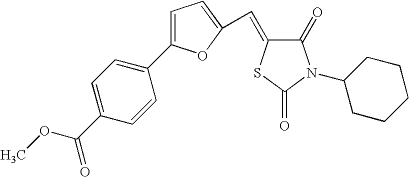 | less active | 4-[5-(3-cyclohexylmethyl-2,4- dioxo-thiazolidin-5- ylidenemethyl)-furan-2-yl]- benzoic acid methyl ester |
| 33 | 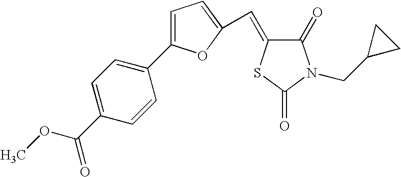 | less active | 4-[5-(3-cyclopropylmethyl-2,4- dioxo-thiazolidin-5- ylidenemethyl)-furan-2-yl]- benzoic acid methyl ester |
| 34 | 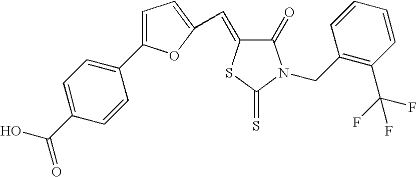 | 1.5 | 4-{5-[4-oxo-2-thioxo-3-(2- trifluoromethyl-benzyl)- thiazolidin-5-ylidenemethyl]- furan-2-yl}-benzoic acid |
| 35 | 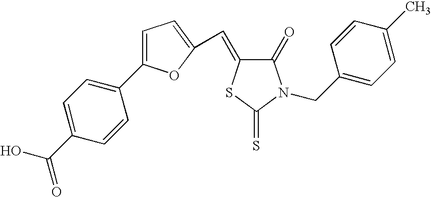 | 2 | 4-{5-[3-(4-methyl-benzyl)-4- oxo-2-thioxo-thiazolidin-5- ylidenemethyl]-furan-2-yl}- benzoic acid |
| 36 | 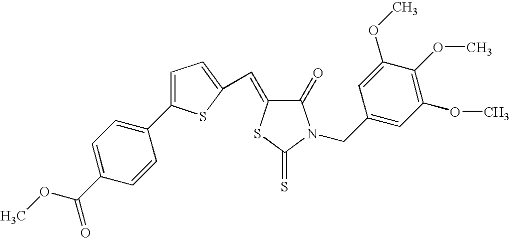 | less active | 4-{5-[4-oxo-2-thioxo-3-(3,4,5- trimethoxy-benzyl)-thiazolidin- 5-ylidenemethyl]-thiophen-2- yl}-benzoic acid methyl ester |
| 37 | 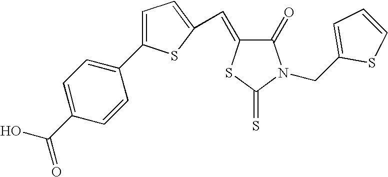 | 5 | 4-[5-(4-oxo-3-thiophen-2- ylmethyl-2-thioxo-thiazolidin-5- ylidenemethyl)-thiophen-2-yl]- benzoic acid |
| 38 | 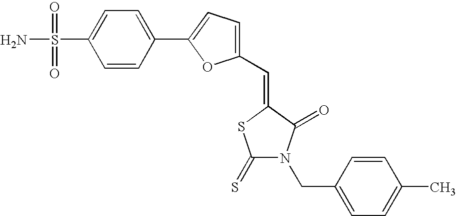 | 6 | 4-{5-[3-(4-methyl-benzyl)-4- oxo-2-thioxo-thiazolidin-5- ylidenemethyl]-furan-2-yl}- benzenesulfonamide |
| 39 | 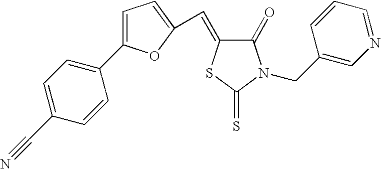 | 4 | 4-[5-(4-oxo-3-pyridin-3- ylmethyl-2-thioxo-thiazolidin-5- ylidenemethyl)-furan-2-yl]- benzonitrile |
| 40 | 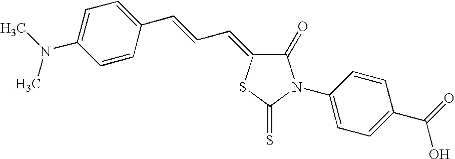 | 8 | 4-{5-[3-(4-dimethylamino- phenyl)-allylidene]-4-oxo-2- thioxo-thiazolidin-3-yl}-benzoic acid |
| 41 | 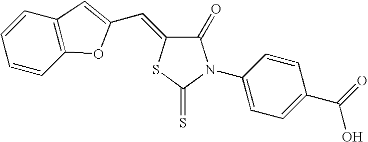 | 2 | 4-(5-benzofuran-2-ylmethylene- 4-oxo-2-thioxo-thiazolidin-3-yl)- benzoic acid |

Preferred bi- and tricyclic thiazolidinones are those of the Formula II:

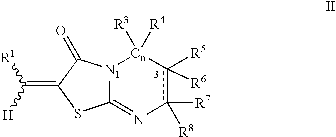

wherein the dotted line represents a double or a single bond; R¹ is alkenyl, aryl or heteroaryl; R³ and R⁴ are each independently hydrogen, alkyl or aryl; R⁵, R⁶, R⁷ and R⁸ are each independently hydrogen, alkyl, acyl or aryl; or R⁵ and R⁷, combined, form an aryl, heteroaryl, cycloalkyl or heteroalicyclic ring; and n is 0 or 1; or pharmaceutically acceptable salts, compositions and prodrugs thereof;

with the proviso that:

(a) when n is 0, R³ and R⁴ are absent; and

(b) when the dotted line represents a double bond, R⁵ and R⁷ are absent.

For the sake of clarity, it should be noted that when n is 0, a double bond between the carbon atom bearing R⁵ and R⁶ and the carbon atom bearing R⁷ and R⁸ is absent. A preferred R¹ is heteroaryl. An example of particularly preferred R¹ substituents is aryl substituted furanyl. Such an aryl substituted furanyl includes carboxyphenyl substituted furanyl. The carboxylic acid group may be replaced, for example, with ester, sulfonamide, aminosulfonyl or tetrazole. Additional preferred R¹ substituents include those set forth above as preferred for R¹ of Formula I (monocyclic). Preferred R³-R⁸ substituents include those set forth above as preferred for R² of Formula I (monocyclic).

Preferred bicyclic thiazolidinones of the Formula II of the preferred embodiments of the present invention are shown in Table 2.

TABLE 2

| | | IC₅₀ | |
|---|---|---|---|
| | Compound | (μM) | Compound Name |
| 42 | 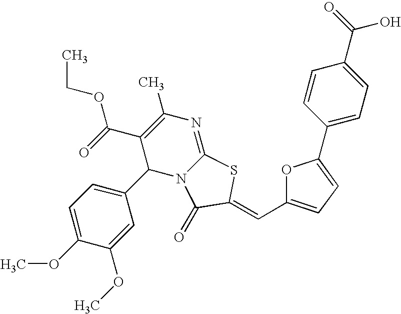 | 4.5 | 2-[5-(4-Carboxy-phenyl)-furan-2- ylmethylene]-5-(3,4-dimethoxy-phenyl)- 7-methyl-3-oxo-2,3-dihydro-5H- thiazolo[3,2-a]pyrimidine-6-carboxylic acid ethyl ester |
| 43 | 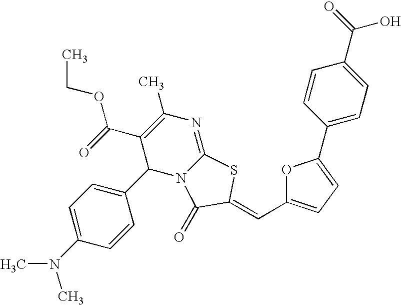 | 2.7 | 2-[5-(4-Carboxy-phenyl)-furan-2- ylmethylene]-5-(4-dimethylamino- phenyl)-7-methyl-3-oxo-2,3-dihydro- 5H-thiazolo[3,2-a]pyrimidine-6- carboxylic acid ethyl ester |
| 44 | 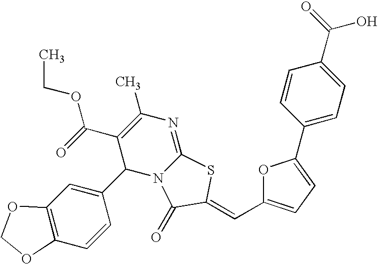 | 2.3 | 5-Benzo[1,3]dioxol-5-yl-2-[5-(4- carboxy-phenyl)-furan-2-ylmethylene]- 7-methyl-3-oxo-2,3-dihydro-5H- thiazolo[3,2-a]pyrimidine-6-carboxylic acid ethyl ester |
| 45 | 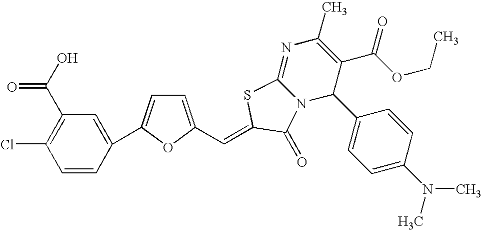 | 1.9 | 2-[5-(3-Carboxy-4-chloro-phenyl)- furan-2-ylmethylene]-5-(4- dimethylamino-phenyl)-7-methyl-3-oxo- 2,3-dihydro-5H-thiazolo[3,2- a]pyrimidine-6-carboxylic acid ethyl ester |
| 46 | 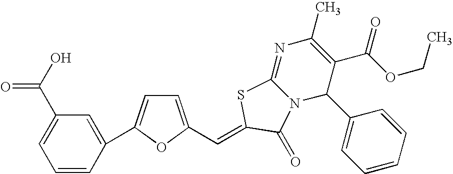 | 4.3 | 2-[5-(3-Carboxy-phenyl)-furan-2- ylmethylene]-7-methyl-3-oxo-5-phenyl- 2,3-dihydro-5H-thiazolo[3,2- a]pyrimidine-6-carboxylic acid ethyl ester |
| 47 | 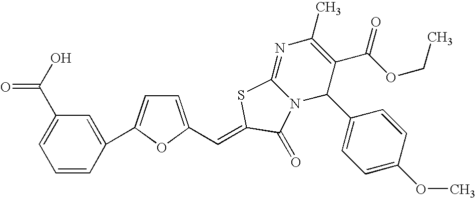 | 2 | 2-[5-(3-Carboxy-phenyl)-furan-2- ylmethylene]-5-(4-methoxy-phenyl)-7- methyl-3-oxo-2,3-dihydro-5H- thiazolo[3,2-a]pyrimidine-6-carboxylic acid ethyl ester |
| 48 | 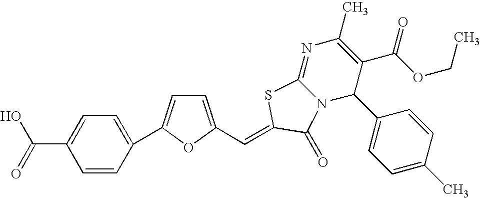 | 3.5 | 2-[5-(4-Carboxy-phenyl)-furan-2- ylmethylene]-7-methyl-3-oxo-5-p-tolyl- 2,3-dihydro-5H-thiazolo]3,2- a]pyrimidine-6-carboxylic acid ethyl ester |
| 49 | 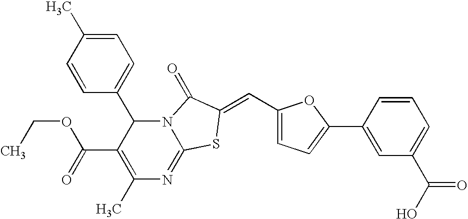 | 2.8 | 2-[5-(3-Carboxy-phenyl)-furan-2- ylmethylene]-7-methyl-3-oxo-5-p-tolyl- 2,3-dihydro-5H-thiazolo[3,2- a]pyrimidine-6-carboxylic acid ethyl ester |
| 50 | 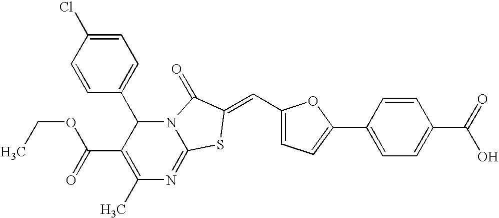 | 3.4 | 2-[5-(4-Carboxy-phenyl)-furan-2- ylmethylene]-5-(4-chloro-phenyl)-7- methyl-3-oxo-2,3-dihydro-5H- thiazolo[3,2-a]pyrimidine-6-carboxylic acid ethyl ester |
| 51 | 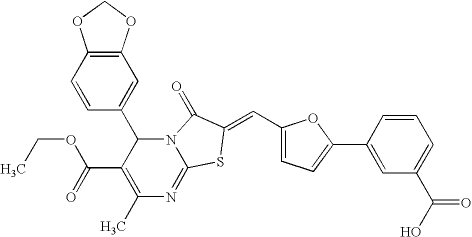 | 2.7 | 5-Benzo[1,3]dioxol-5-yl-2-[5-(3- carboxy-phenyl)-furan-2-ylmethylene]- 7-methyl-3-oxo-2,3-dihydro-5H- thiazolo[3,2-a]pyrimidine-6-carboxylic acid ethyl ester |
| 52 | 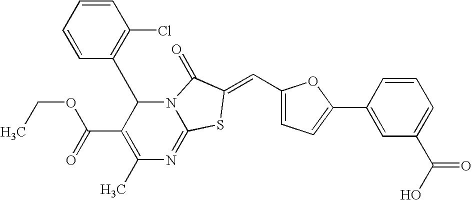 | 4 | 2-[5-(3-Carboxy-phenyl)-furan-2- ylmethylene]-5-(2-chloro-phenyl)-7- methyl-3-oxo-2,3-dihydro-5H- thiazolo[3,2-a]pyrimidine-6-carboxylic acid ethyl ester |
| 53 | 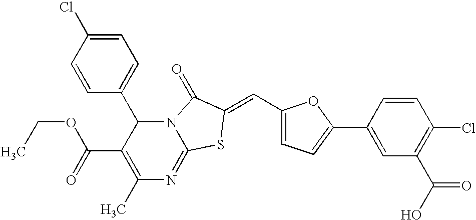 | 2.5 | 2-[5-(3-Carboxy-phenyl)-furan-2- ylmethylene]-5-(4-chloro-phenyl)-7- methyl-3-oxo-2,3-dihydro-5H- thiazolo[3,2-a]pyrimidine-6-carboxylic acid ethyl ester |
| 54 | 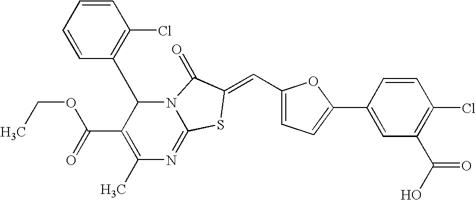 | 4.4 | 2-[5-(3-Carboxy-phenyl)-furan-2- ylmethylene]-5-(2-chloro-phenyl)-7- methyl-3-oxo-2,3-dihydro-5H- thiazolo[3,2-a]pyrimidine-6-carboxylic acid ethyl ester |
| 55 | 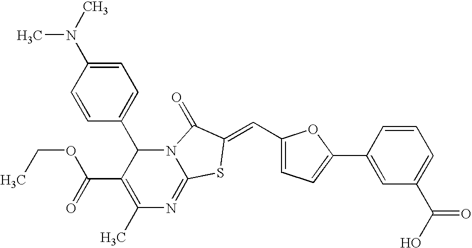 | 0.7 | 2-[5-(3-Carboxy-phenyl)-furan-2- ylmethylene]-5-(4-dimethylamino- phenyl)-7-methyl-3-oxo-2,3-dihydro- 5H-thiazolo[3,2-a]pyrimidine-6- carboxylic acid ethyl ester |
| 56 | 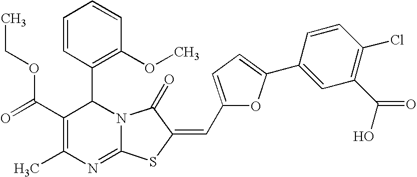 | 2.6 | 2-[5-(3-Carboxy-4-chloro-phenyl)- furan-2-ylmethylene]-5-(2-methoxy- phenyl)-7-methyl-3-oxo-2,3-dihydro- 5H-thiazolo[3,2-a]pyrimidine-6- carboxylic acid ethyl ester |
| 57 | 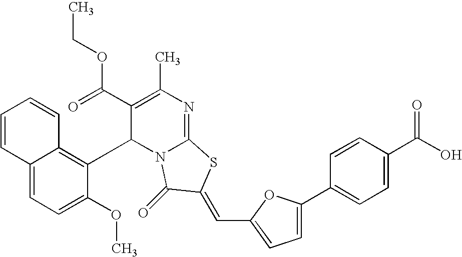 | 3.7 | 2-[5-(4-Carboxy-phenyl)-furan-2- ylmethylene]-5-(2-methoxy-naphthalen- 1-yl)-7-methyl-3-oxo-2,3-dihydro-5H- thiazolo[3,2-a]pyrimidine-6-carboxylic acid ethyl ester |
| 58 | 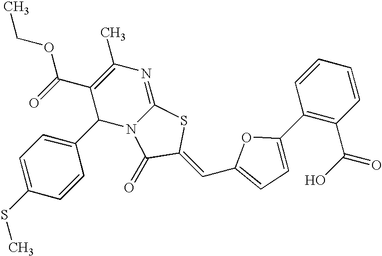 | 4 | 2-[5-(2-Carboxy-phenyl)-furan-2- ylmethylene]-7-methyl-5-(4- methylsulfanyl-phenyl)-3-oxo-2,3- dihydro-5H-thiazolo[3,2-a]pyrimidine- 6-carboxylic acid ethyl ester |
| 59 | 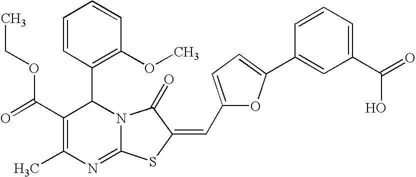 | 4.1 | 2-[5-(3-Carboxy-phenyl)-furan-2- ylmethylene]-5-(2-methoxy-phenyl)-7- methyl-3-oxo-2,3-dihydro-5H- thiazolo[3,2-a]pyrimidine-6-carboxylic acid ethyl ester |

Preferred tricyclic thiazolidinones of the Formula II of the preferred embodiments of the present invention are shown in Table 3.

TABLE 3

| | | IC₅₀ | |
|---|---|---|---|
| | Compound | (μM) | Compound Name |
| 60 | 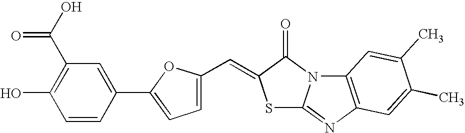 | 3.6 | 5-[5-(6,7-Dimethyl-3-oxo- benzo[4,5]imidazo[2,1-b]thiazol-2- ylidenemethyl)-furan-2-yl]-2-hydroxy- benzoic acid |
| 61 | 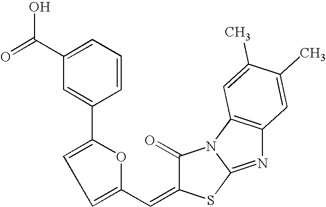 | 3.4 | 3-[5-(6,7-Dimethyl-3-oxo- benzo[4,5]imidazo[2,1-b]thiazol-2- ylidenemethyl)-furan-2-yl]-benzoic acid |

II. PLD and Proliferative Diseases

As noted above, lipid-derived metabolites play an important role in the regulation of cell responses to external stimuli, including cell growth control, transformation and apoptosis. Phospholipase D (PLD) is one of the critical elements in the regulation of lipid metabolism and the generation of second messengers, some of them involved in cell growth control. Lucas et al., Oncogene 19:431 (2000). A common response to oncogenic and mitogenic stimuli is elevated PLD activity. RalA, a small GTPase that functions as a downstream effector molecule of Ras, exists in a complex with PLD1. Lu et al., Mol. Cell Biol. 20:462 (2000). Workers in the field have examined the PLD activities of human renal cancers and found that the PLD2 activity was greatly elevated in almost all cases examined as compared with the adjacent normal region. Zhao et al., Biochem. Biophys. Res. Commun. 278:140 (2000). Noh et al. studied the expression of PLD in human breast carcinomas and non-malignant tissues and found that PLD protein and mRNA levels were overexpressed in 14 of 17 breast cancer tissues. Noh et al., Cancer Lett. 161:207 (2000). Fiucci et al. have found that phospholipase D (PLD), a constituent enzyme of caveolae and detergent-insoluble glycolipid-rich membranes (DIGs), is up-regulated in human multi-drug resistance (NIDR) cancer cells. Fiucci et al., Int. J. Cancer 85:882 (2000).

The preferred embodiments of the present invention contemplate generally a method for inhibiting PLD (phospholipase D), comprising contacting PLD in vivo or in vitro with an amount of a thiazolidinone effective to inhibit PLD activity. It is clear from the discussion above, that PLD activity is elevated in the cancers discussed above. It should also be mentioned that elevated PLD activity has also been implicated in a variety of cellular processes, including inflammation. Kim et al., J. Immunol. 163:5462-70 (1999); Gomez-Munoz et al., J Lipid Res. 40:988-93 (1999). While not wishing to be bound by theory, it is believed that PLD causes a sustained activation of protein kinase C (PKC) through the primary production of phosphatidic acid from phosphatidylcholine. Phosphatidic acid in turn is dephosphorylated forming diacylglycerol. It is known that phosphatidic acid is metabolically convertible to the mitogenic lipids diacylglycerol and lysophosphatidic acid. Uchida et al., J. Cancer Res. Clin. Oncol. 123:280 (1997). Further, diacylglycerol is a well known activator of PKC (Takai et al., Biochem. Biophys. Res. Commun. 91:1218 (1979)) and lysophosphatidic acid is a potent mitogen for cultured cells (Van Corven et al., Cell 59:45 (1989)), and it promotes tumor cell invasion in vitro. Imamura et al., Biochem. Biophys. Res. Commun 193:497 (1993).

It should be noted that modulators of PKC have been shown to have both antiproliferative and anti-inflammatory effects in vitro. Some anti-psoriasis drugs, such as cyclosporine A and anthralin, have been shown to modulate PKC. Modulators of PKC have been suggested as a therapeutic approach to the treatment of psoriasis. Hegemann and Mahrle in PHARMACOLOGY OF THE SKIN 357-68, H. Mukhtar, ed. (CRC Press, 1992). Conversely, PLD1 or PLD2 can also be activated by PKC. Mwanjewe et al., Biochem. J. 359:211 (2001). While compounds of the preferred embodiments of the present invention are not per se inhibitors of PKC, they may be considered indirect modulators of that enzyme. Thus, proliferative and inflammatory diseases may be treated by modulating PKC via the inhibition of PLD.

The compounds of the embodiments of the present invention can be useful in treating diseases associated with PLD and, indirectly, those associated with PKC, as well. This group of diseases encompasses: hyperproliferative and inflammatory conditions, including psoriasis, and various cancers, such as bladder cancer, skin cancer, breast cancer, renal cancer, lung cancer, colon cancer, and human multi-drug resistance cancer.

Thus, the compounds of the embodiments of the present invention are contemplated as PLD inhibitors generally, and in particular for the treatment of cancer and inflammation. One aspect of the present invention, therefore, encompasses the treatment of cancer by administering to a subject in need thereof an effective amount either of a thiazolidinone or a pharmaceutically acceptable salt or composition or prodrug thereof, thereby for example inhibiting cell proliferation.

In the context of the embodiments of the present invention, the term “subject” refers to any animal, including humans and other primates, rodents (e.g., mice, rats, and guinea pigs), lagamorphs (e.g., rabbits), bovines (e.g., cattle), ovines (e.g., sheep), caprines (e.g., goats), porcines (e.g., swine), equines (e.g., horses), canines (e.g., dogs), felines (e.g., cats), domestic fowl (e.g., chickens, turkeys, ducks, geese, other gallinaceous birds, etc), as well as feral or wild animals, including such animals as ungulates (e.g., deer), bear, fish, lagamorphs, rodents, birds, etc. It is not intended that the term be limited to a particular age or sex. Thus, adult and newborn subjects, as well as fetuses, whether male or female, are encompassed by the term.

III. Pharmaceutical Compositions: Therapeutic and Other Applications

A compound within the embodiments of the present invention can be given per se to a patient, or it can be administered in pharmaceutical compositions that also contain a pharmaceutically acceptable carrier, diluent or excipient, inter alia. Techniques for formulation and administration of drugs may be found in the latest edition of REMINGTON'S PHARMACEUTICAL SCIENCES, Mack Publishing Co. (Easton, Pa.).

A. Routes of Administration

Suitable routes of administration may include parenteral, oral, rectal, transmucosal or intestinal administration or intramuscular, subcutaneous, intramedullary, intrathecal, direct intraventricular, intravenous, intraperitoneal or intranasal injections.

Alternately, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

B. Composition/Formulation

Pharmaceutical compositions of the compounds and the pharmaceutically acceptable salts thereof are preferred embodiments of this invention. Pharmaceutical compositions of the embodiments of the present invention may be manufactured by processes well known in the art; e.g., by means of conventional mixing, dissolving, granulating, dragee-making, levigating, emulsifying, encapsulating, entrapping or lyophilizing processes.

Pharmaceutical compositions for use in accordance with the embodiments of the present invention thus may be formulated in a conventional manner using one or more pharmaceutically acceptable carriers, diluents, excipients, and/or auxiliaries which facilitate processing of the active compounds into preparations which can be used pharmaceutically. Proper formulation is dependent upon the route of administration chosen. The thiazolidinones of the embodiments of the present invention may be formulated such that the formulation comprises a single thiazolidinone or a mixture of one or more of the types of thiazolidinones described here.

For injection, the compounds of the invention may be formulated as sterile aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be made with the use of a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the embodiments of the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoro-ethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions for parenteral administration include sterile aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation (see, for example, U.S. Pat. No. 5,702,717 for a biodegradable depot for the delivery of a drug). Such long acting formulations may be administered by implantation (for example subcutaneously or intramuscularly) or by intramuscular injection. Thus, for example, the compounds may be -formulated with suitable polymeric or hydrophobic materials (for example as an emulsion in an acceptable oil) or ion exchange resins, or as sparingly soluble derivatives, for example, as a sparingly soluble salt. The pharmaceutical compositions herein also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compounds of the invention that modulate PLD may be provided as pharmaceutically acceptable salts wherein the claimed compound may form the negatively or the positively charged species. Examples of salts in which the compound forms the positively charged moiety include, without limitation, quaternary ammonium (defined elsewhere herein), salts such as the hydrochloride, sulfate, carbonate, lactate, tartarate, maleate, succinate, etc. formed by the reaction of an amino group with the appropriate acid.

C. Dosage

Pharmaceutical compositions suitable for use in the embodiments of the present invention include compositions wherein the thiazolidinones are contained in a therapeutically effective amount which achieves inhibition of PLD sufficient for the particular purpose.

More specifically, the term “therapeutically effective amount” as used herein refers to that amount of the compound being administered which will relieve to some extent one or more of the symptoms of the disorder being treated. In reference to the treatment of cancer, a therapeutically effective amount refers to that amount that has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth, (4) relieving to some extent (or, preferably, eliminating) one or more symptoms associated with the cancer, and/or (5) prolonging the survival time of the recipient. In reference to the treatment of inflammation, a therapeutically effective amount refers to the amount that has the effect of (1) reduction of histamine and leukotriene releases during mast cell activation [Ro et al., J. Pharmacol. Exp. Ther. 292:114-121, 2000], (2) inhibition of up-regulation of cytosolic Phospholipase A2 and Prostaglandin E2 levels after treatment of macrophages with lipopolysaccharide, interleukin-1beta, or tumor necrosis factor-alpha [Ribardo et al., J. Biol. Chem. 276:5467-5475, 2001], (3) reduction of mucosal damage in colitis induced by reactive oxygen metabolites [Sakamoto et al., J. Gastroenterol. Hepatol. 15:1138-1144, 2000], (4) reduction of tissue damage due to production of superoxide anions through activation of leukotriene B4 receptor in neutrophils [Levy et al., Ann. N.Y. Acad. Sci. 905:69-80, 2000], (5) reduction of neutrophils activation and superoxide generation in response to IL-8 [Brandolini et al., J. Leukoc. Biol. 59:427-434, 1996], and/or (6) reduction of the symptoms associated with inflammatory diseases such as allergy, asthma, arthritis, psoriasis, dermatitis, and colitis.

Determination of a therapeutically effective amount is well within the capability of those of ordinary skill in the art, especially in light of the detailed disclosure provided herein.

For any compound used in the methods of the invention, the therapeutically effective amount or dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of PLD activity). Such information can be used to more accurately determine useful doses in humans.

Toxicity and therapeutic efficacy of the compounds described herein can be determined by standard pharmaceutical procedures in cell cultures or experimental animals for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration, and dosage can be chosen by the individual physician in view of the patient's condition. For example, see Fingl et al. in 1 THE PHARMACOLOGICAL BASIS OF THERAPEUTICS (1975).

Dosage amount and interval may be adjusted individually, to provide plasma levels of the active moiety that are sufficient to maintain PLD modulatory effects, or minimal effective concentration (MEC). The MEC will vary for each compound but can be estimated from in vitro data; for example, the concentration necessary to achieve 50-90% modulation of PLD, using the assays described herein. Dosages necessary to achieve the MEC will depend on individual characteristics and route of administration. In any event, HPLC assays or bioassays can be used to determine plasma concentrations.

Dosage intervals also can be determined using MEC value. Compounds should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician. An exemplary systemic daily dosage is about 5 to about 200 mg/kg of body weight. Normally, from about 10 to about 100 mg/kg of body weight of the compounds of the preferred embodiments of the preferred embodiments of the present invention, in one or more dosages per day, is effective to obtain the desired results. One of ordinary skill in the art can determine the optimal dosages and concentrations of the compounds of the embodiments of the present invention with only routine experimentation.

The compounds of the embodiments of the present invention are substantially pure and preferably sterile. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

D. Other Uses

The compounds of the embodiments of the present invention may be employed not only for therapeutic purposes, but also as aids in performing research in vitro. For example, the compounds of the embodiments of the present invention may be used to study biochemical pathways that would require the modulation of PLD to give elevated or decreased levels of PA relative to a predetermined control. Modulation of PLD may result in the prolonged or limited activity of biochemical pathways that depend on, or respond to, increased or decreased levels of PA.

Additionally, a cell culture medium comprising the compounds of the embodiments of the present invention is within the scope of the invention.

The following examples are offered by way of illustration and not by way of limitation.

### EXAMPLES

### Example 1

### Construction of Expression Plasmids for PLD1 and PLD2

Recombinant PLD1 and PLD2—Human PLD1 and PLD2 were isolated from Sf9 insect cells that had been infected with recombinant baculovirus vector, pFastBac™HTc vector (Life Technologies, Gaithersburg, Md.), containing PLD1 or PLD2 cDNAs.

PLD1 cDNA was isolated from a human liver cDNA library (Life Technologies, Gaithersburg, Md.). Search of the Genbank with the blastp program using the castor bean PCPLD protein sequences as probe came up with a yeast DNA sequence (Genbank Accession# Z28256) that encodes a S. cerevisiae reading frame (ORF YKR031c) mapped to chromosome XI. Wang et al., J. Biol. Chem. 269:20312 (1994). This sequences contain several short stretches of amino acids homology to the plant PCPLD protein sequence. This yeast sequence has been identified to encode the SP014 gene (Genbank Accession# L46807), a gene essential for yeast meiosis. Honigberg et al., Genetics 130:703 (1992). The gene product for SP014 has been found to contain PCPLD activity. Ella et al., Biochem. J. 314:15 (1996); Rose, et al., Proc. Nat'l Acad. Sci. 92:12151 (1995). This yeast protein sequence was then used to search for homologous sequences in the Genbank database of expressed sequence tags (dbEST). Several short sketches of human cDNA sequences with homology to the plant PCPLD and the yeast SP014 protein sequence were found.

Accordingly, synthetic oligonucleotides 5′-GTCCATGCTA ATGTACAGTT GCTC -3′ (o. 204986.1), was synthesized (Life Technologies, Gaithersburg, Md.) based on the putative coding sequence of the human cDNA clone genbank#204986, in which one nucleotide was changed from C to T to generate a BsrG I site. o.204986.1 was used in combination with the primer 5′-CTAGCTTATA ATACGACTCA C-3′ (o.sport.1R) corresponding to the vector sequence just downstream of the cDNA cloning region of the plasmid pCMV.SPORT (Life Technologies, Gaithersburg, Md.) to isolate the 3′-region of the human PCPLD cDNA from a human liver cDNA library (Life Technologies, Gaithersburg, Md.) using Expand™ long template PCR (Boehringer Mannheim, Indianapolis, Ind.). A 1,300 bp and a 900 bp PCR fragments were generated. These two fragments were cleaved with BsrG I and Xho I prior to subcloning into the Litmus28 vector (New England Biolab, Beverly, Mass.) between the Acc65 I and the Xho I site. DNA sequence analysis showed that the open reading frame of the cDNA clone with the 1,300 bp insert, pL28.Li.29, matched perfectly with aa 742-1074 of the human PCPLD (hPLD1) sequence, whereas only the first 650 by of the cDNA clone with the 900 bp insert matched with the hPLD1 sequence and with divergence of the coding sequence after aa 961, suggesting that this cDNA clone, pL28.Li.8, represented an alternatively spliced variant of hPLD1 encoding a protein with a different C-terminal sequence. Hammond et al., J. Biol. Chem. 270:29640 (1995).

To isolate the 5′-region of the human PCPLD cDNA from a human liver cDNA library (Life Technologies, Gaithersburg, Md.), using Expand™ long template PCR (Boehringer Mannheim, Indianapolis, Ind.), a primer 5′-TTCCCTGTGA GCTTTCAGGA TCCT-3′ (o.pld1.R) complementary to the region corresponding to aa 804-810 of hPLD1 was used in combination with the primer 5′-CGCCAACGC GAGGTGCTAG C-3′ (o.pld1.1F) corresponding to the region near the Nhe I site in the 5′-untranslated region of hPLD1. The PCR fragments generated were cleaved with Nhe I and BamH I. The fragments with sizes of about 2,400 bp were isolated from agarose gel prior to subcloning into the pLitmus38 vector (New England Biolab, Beverly, Mass.). DNA sequence analysis showed the cDNA clone, pL38.1.6, with a 2,450 bp Nhe I-BamH I insert contained a open reading frame with perfect match to amino acids 1-805 of hPLD1, whereas the cDNA clone, pL38.1.4, with a 2,350 bp Nhe I-BamH I insert matched perfectly with an alternatively spliced variant of hPLD1, known as PLD1b with a 38 aa deletion in the region corresponding to aa 585-622 of hPLD1. Hammond et al., J. Biol. Chem. 272:3860 (1997).

To assemble the various hPLD1 isoforms, the following fragments were isolated:

1) The 2,500 bp BsrG I-BamH I fragment from pL38.1.6.

2) The 2,400 bp BsrG I-BamH I fragment from pL38.1.4.

3) The 1,130 bp BamH I (partially digested)-Not I fragment from pL28.Li.29.

4) The 662 bp BamH I-Not I fragment from pL28.Li.8.

Fragments 1 and 3 were inserted via a three-part ligation into pBluescriptSK(−)II cleaved with Acc65 I and Not I to generate pskPLD1.6. Fragments 2 and 3 were inserted via a three-part ligation into pBluescriptSK(−)II cleaved with Acc65 I and Not I to generate pskPLD1.4. Fragments 1 and 4 were inserted via a three-part ligation into pBluescriptSK(−)II, cleaved with Acc65 I and Not I to generate pskPLD1.5.

For the expression of the various hPLD1 isoforms in mammalian cells, the 3,600 bp Nhe I-Not I fragment from pskPLD1.6., the 3,500 bp Nhe I-Not I fragment from pskPLD1.4., and the 3,100 bp Nhe I-Not I fragment from pskPLD1.5. were inserted, respectively, into the pCE2 vector cleaved with Nhe I and Not I to generate pC2PLD1.6, pC2PLD1.4, and pC2PLD1.5.

The plasmid pCE2 was derived from pREP7b with the RSV promoter region replaced by the CMV enhancer and the elongation factor-1α (EF-1α) promoter and intron. Leung et al., Proc. Nat'l Acad. Sci. USA 92:4813 (1995). The CNN enhancer came from a 380 bp Xba I-Sph I fragment produced by PCR from pCEP4 (Invitrogen, San Diego, Calif.) using the primers 5′-GGCTCTAGAT ATTAATAGTA ATCAATTAC-3′ and 5′-CCTCACGCAT GCACCATGGT AATAGC-3′. The EF-1α promoter and intron came from a 1200 bp Sph I-Asp7 18 I fragment produced by PCR from human genomic DNA using the primers 5′-GGTGCATGCG TGAGGCTCCG GTGC-3′ and 5′-GTAGTTTTCA CGGTACCTGA AATGGAAG-3′. Uetsuki et al., J. Biol. Chem. 264:5791 (1989). These two fragments were ligated into a Xba I/Asp718 I digested vector derived from pREP7b to generate pCE2.

For the construction of Baculovirus expression vectors, the various hPLD1 isoforms were amplified by PCR from the DNA templates pskPLD1.6, pskPLD1.4, and pskPLD1.5 using the primers 5′-GCATGTCGAC CTCACTATAG GGCGAATTGG-3′ (opldx14F)and 5′-GCATTCTAGA GCTGGAGCTC CACCGCGGTG-3′ (opldx12R). The fragments generated were cleaved with Sal I and Xba I for insertion into pFastBac™HTc vectors (Life Technologies, Gaithersburg, Md.) between the Sal I and Xba I sites for the generation of the plasmids pFB.PLD1.6, pFB.PLD1.4, and pFB.PLD1.5. These plasmids were then transformed into E. coli DH10Bac (Life Technologies, Gaithersburg, Md.) for the generation of recombinant Bacmid DNA for transfection into HighFive (Invitrogen, San Diego, Calif.) or Sf9 insect cells for the production of recombinant Baculovirus stocks. pFB.PLD1.4 was the main plasmid used for the production of PLD1 for use in various enzymatic assays.

A variant hPLD2.2 cDNA (Leung et al., U.S. Pat. No. 6,274,363) was isolated based on the hPCPLD (referred as hPLD2.1 herein) sequence disclosed previously. Leung et al., U.S. Pat. No. 5,859,222. Three overlapping hPLD2.2 cDNA fragments were isolated from a human liver cDNA library (Life Technologies, Gaithersburg, Md.) using the hPLD2.1 cDNA fragment as probe. For the assembling of a full-length hPLD2.2 cDNA clone, the 1,600 bp EcoRI-BstBI fragment, the 660 bp BstBI-BanI fragment, and the 1,145 bp BanI-NotI fragment were ligated into the EcoRI-NotI vector of pBluescriptSK (Stratagene, La Jolla, Calif.) to generate the plasmid pSK.PLD2.2. DNA sequence analysis showed the sequence of PLD2.2 was identical to that of hPLD2.1 with the exceptions of 6 nucleotide changes scattered throughout the entire molecule. Apart from a few nucleotide changes, the sequence of PLD2.2 was essentially identical to other published human PLD2 sequences. Lopez et al., J. Biol. Chem. 273:12846 (1998); Saqib et al., GenBank Accession No. AF038440, Jan. 16, 1998; Steed et al., FASEB J. 12:1309 (1998). All the other human PLD2 sequences also contain a few non-conserved nucleotide changes with respect to one another, suggesting the presence of multiple single nucleotide polymorphism within the PLD2 sequence.

For the construction of hPLD2 Baculovirus expression vectors, the full-length hPLD2.2 cDNA was amplified by PCR from the DNA template pSK.PLD2.2, whereas the full-length hPLD2.1 cDNA was amplified by PCR from the DNA template pCE2.PLD using the primers 5′-GCGCGGAATT CGGTACCAGG TATGACGGCG AC -3′ (opld2_{—}1F)and 5′-GACAAGCTTG CGGCCGCTTA ATTAAAGATCT TTTTTTTTTT T-3′ (opld2_{—}1R). The fragments generated were cleaved with EcoR I and Hind III for insertion into pFastBacl (Life Technologies, Gaithersburg, Md.) between the EcoR I and Hind III sites for the generation of the plasmid pFBPLD2.2 and pFBPLD2.1. These plasmids were then transformed into E. coli DH10Bac (Life Technologies, Gaithersburg, Md.) for the generation of recombinant Bacmid DNA for transfection into HighFive (Invitrogen, San Diego, Calif.) or Sf9 insect cells for the production of recombinant Baculovirus stocks.

pFBPLD2.2 was found to have higher activity than pFBPLD2.1 when expressed in Sf9 cells, suggesting that some of the minor changes in amino acid sequence can affect PLD2 activity. To facilitate purification of PLD2 from Sf9 cell extracts, the PLD2.2 cDNA from pFBPLD2.2 was amplified by PCR using the primers 5′-GCCTACGTCG ACATGACGGC GACCCCTGAG AGC-3′ and 5′-CAGGCTCTAG ACTATGTCCA CACTTCTAGG-3′. The ˜2800 bp fragment generated was cleaved with Sal I and Xba I for insertion into pFastBac™HTc vectors (Life Technologies, Gaithersburg, Md.) between the Sal I and Xba I sites for the generation of the plasmid pFBHcPLD2 used for the expression of PLD2 with a hexa-histidine tag at the amino-terminus. This plasmid, pFBHcPLD2, was then transformed into E. coli DH10Bac, a product of Invitrogen, division of Life Technologies (Gaithersburg, Md.), for the generation of recombinant Bacmid DNA for transfection into HighFive, a product of Invitrogen (San Diego, Calif.) or SF9 insect cells, for the production of recombinant Baculovirus stocks used for the production of PLD2, to use in various enzymatic assays.

1. In Vitro Assay of PLD Activity

PLD1 activity was assayed by measuring the generation of [³H]-choline from [choline-methyl³H](16:0, 16:0)PC. Each sample (100 μl) was made up of lipid vesicles containing PE (552 μM), PIPₓ (92 μM), and PC (92 μM) with 25,000 cpm [choline-methyl³H](16:0, 16:0)PC in a molar ratio of 6:1:1, 50 mM Hepes-NaOH (pH 7.5), 200 mM NaCl, 20 μM guanosine 5′-O-(3′-thiotriphosphate) (GTPγS), cytosol fractions (0.1 mg/ml) from Sf9 cells infected with recombinant viruses, respectively, expressing ADP-ribosylation factor (arf1) and protein kinase c (PKC-α), PLD1 (20 μg/ml) partially purified using a TALON™ metal affinity column (Clontech, Palo Alto, Calif.) from cytosol fractions from Sf9 cells infected with recombinant viruses expressing a PLD1 fusion protein with a polyhistidine tag at the N-terminus, and +/−compound (dissolved in 100% DMSO) of interest with a final concentration of compound at 16 μM and DMSO at 8%.

The assay procedure for PLD2 was similar to that for PLD1, with the exception that arf1, PKC-α, and GTPγS were omitted from the reaction mixture. After addition of enzymes, the reactions were incubated for 1 hour at room temperature. Twenty microliters of 12% trichloroacetic acid (TCA) then were added to each sample, to stop the reaction and to precipitate the unreacted [choline-methyl³H](16:0, 16:0)PC. Thereafter, the samples were passed through 0.65 μm Millipore 96 well filter plates to removed the excess [choline-methyl³H](16:0, 16:0)PC. For each sample the filtrate (50 μl), which contained the [³H]-choline released by PLD cleavage of PC, was analyzed using a TopCount™ Liquid Scintillation Counter, a product of Packard (Meriden, Conn.).

2. Measurement of PLD Activity in Intact Cells

PLD activity in cells was assayed by measuring the accumulation of [1⁴C]phosphatidylbutanol. Various mammalian cell lines (5×10⁵ per well) were incubated in the absence or presence of 16 μM compound in 2 ml of DMEM for 30 min to 48 h (depending on the solubility of the various compounds) prior to labeling by incubation for 2 h with 10 μCi of [¹⁴C]myristic acid. The labeled cells were washed with DMEM, kept in 2 ml of DMEM containing 0.3% butanol in the absence or presence of 100 nM PMA for 20 min. The medium was then removed and replaced with ice-cold PBS to terminate cell stimulation. The cells were scraped into an ice-cold vial and then extracted with a mixture of chloroform and methanol (2:1). The organic phase was dried, redissolved in chloroform and methanol (2:1), and spotted onto Silica Gel 60 thin layer chromatography plates (Merck). The labeled compounds were separated by a multistep thin-layer chromatography (TLC) (White et al., Anal. Biochem. 258:109 (1998)), using the solvent CHCl₃, CH₃OH, NH₄OH (70:25:3), for developing the first quarter of the plate, and then the solvent CHCl₃, CH₃OH, NH₄OH (90:10:1), for the rest of the plate. The phosphatidylbutanol spots were detected by the Storm PhosphorImager (Molecular Dynamics, Sunnyvale, Calif.). The amount of radioactivity associated with these spots was normalized on the basis of the radioactivity incorporated into total lipids of the cells and was then used as the measure of PLD activity.

The effect of three titratable PLD1 inhibitors on endogenous PLD activity in three pancreatic tumor cell lines was then studied. The three cell lines were preincubated with compounds for 24 hr prior to stimulation with PMA. The measure of inhibition of in-cell PLD activity can be used as a secondary screen for compounds that are active in cells.

To assess whether addition of PLD inhibitors to cells would affect proliferation, a common phenotypic change associated with cancer cells due to defects in controlling mechanism that govern normal cell proliferation and homeostasis (Hanahan et al., Cell 100:57 (2000)), the growth characteristics of non-transformed Rat1 cells and H-ras transformed Rat1 (H-ras Me12/+) cells (Finney et al., Science 260:1524 (1993)) were compared at various concentration of PLD inhibitors. It was shown that 10 μM of one of the PLD inhibitors is sufficient to inhibit the proliferation of the transformed H-ras Me12/+ cells, whereas 25 μM of the inhibitor is required to inhibit the proliferation of the non-transformed Rat1 cells. To confirm whether the compound has a differential effect on transformed cells, a co-culture experiment was set up by plating 200 H-ras Me12/+ cells along with 10⁵ Rat1 cells in 6-well plates. The non-transformed Rat1 cells would grow as a contact-inhibited, adherent monolayer, whereas the transformed H-ras Me12/+ cells would not be contact-inhibited and could keep on growing and piling on top of one another to form foci in the midst of a confluent monolayer of cells. It was found that the compound at ≧20 μM can significantly inhibit foci formation and therefore suppress the propensity for proliferation of H-ras Me12/+ cells.

To determine if the observation of the compound on H-ras Me12/+ cells can be extended to other cell types, the effect of the compound on the proliferation of two human pancreatic tumor cell lines were compared using a clonogenic assay with 150 cells plated per well initially. It was found that the compound at 25 μM can significantly inhibit the capacity for human pancreatic tumor MiaPaCa cells and Panc1 cells to form colonies.

Another common division abnormality of cancer cells is their capacity to grow with less anchorage dependency and to divide when held in suspension without attachment to a rigid surface. This can be assayed by the propensity of a given cell line to form colonies in soft agar, as the presence of soft agar at the bottom of the culture dish eliminates cell contact with the tissue culture plate surface conducive for cell attachment. Panc1 cells can form colonies in soft agar. Panc1 cells in the presence of the compound at 18 μM have greatly reduced capacity to form colonies when seeded at similar numbers. This again suggests that the compound decreases the efficiency of colony formation of Panc1 tumor cells, most likely through inhibition of proliferation.

To determine whether administration of PLD inhibitor would have any effect on tumorgenesis in mice, aliquots of 10⁶ NIH/3T3 cells overexpressing the oncogene Ki-ras were injected intramuscularly into nude mice. The compound at doses that range from 10 mg/kg to 62 mg/kg of mouse body weight was given intra-peritoneally on day 3, 4, 5 and 8 after injection of tumor cells. It was shown that the volume of the tumors in mice treated with the compound were significantly decreased after day 10 when compared to the vehicle control. It is clear that administration of this compound is efficacious in delaying the growth of this highly aggressive NIH/3T3/Ki-ras tumor growth in nude mice. This effect appears to be tumor-static rather than tumor-toxic.

A second compound, with a dimethylxanthine functional moiety, similarly inhibits proliferation of NIH/3T3/Ki-ras cell proliferation in vitro without cytotoxicity using Cyquant and Sytox assays (Molecular Probes, Eugene, Oreg.). Mice are able to tolerate all concentrations and doses (10 mg/kg to 100 mg/kg×5 doses) tested given intra-peritoneally once per day. This compound delays the growth of the highly aggressive NIH/3T3+Ki-ras tumor growth in nude mice at ≧10 mg/kg. This effect appears to be tumor-static rather than tumor-toxic.

### Example 2

### 4-{5-[4-Oxo-2-thioxo-3-(3,4,5-trimethoxy-benzyl)-thiazolidin-5-ylidenemethyl]-furan-2-yl}-benzoic Acid Methyl Ester

A. 4-(5-Formyl-furan-2-yl)-benzoic Acid Methyl Ester

To a solution of 5-bromofuraldehyde (2.43 g, 13.9 mmol), 4-(methoxycarbonyl)phenyl boronic acid (2.50 g, 13.9 mmol), tris(dibenzylideneacetone)dipalladium(0) (192 mg, 0.21 mmol) and potassium fluoride (2.42 g, 41.7 mmol) in 1,4-doxane (100 ml) was added a solution of tri-t-butylphosphine in hexane (10 weight %, 1.01 g, 0.5 mmol). After heating at 65-70° C. for 4 hours, the mixture was cooled to room temperature and treated with dichloromethane (150 ml). After stirring for 10 minutes, the mixture was filtered through a pad of celite and the filtrate was concentrated under reduced pressure. The residue was purified by flash chromatography on silica gel eluting with ethyl acetate-hexane (1:1) to provide 4-(5-formyl-furan-2-yl)-benzoic acid methyl ester (2.6 g, 81% yield).

B. 4-(5-Formyl-furan-2-yl)-benzoic Acid

To a solution of 4-(5-formyl-furan-2-yl)-benzoic acid methyl ester (230 mg, 1 mmol) in a mixture of methanol (6 ml) and water (2 ml) was added lithium hydroxide (82 mg, 2 mmol). After stirring for 6 hours at room temperature, the solution was acidified to pH 2 by addition of 1 M hydrochloric acid. After concentrating under reduced pressure, the residue was purified by flash chromatography on silica gel eluting with 10% methanol-dichloromethane to give 4-(5-formyl-furan-2-yl)-benzoic acid (198 mg, 92% yield).

C. 2-Thioxo-3-(3,4,5-trimethoxy-benzyl)-thiazolidin-4-one

A suspension of bis(carboxymethyl) trithiocarbonate (1.24 g, 5.5 mmol), potassium carbonate (345 mg, 2.5 mmol) and 3,4,5-trimethoxybenzyl amine (986 mg, 5 mmol) in water (20 ml) was heated at reflux for 12 hours. After cooling to room temperature, water (10 ml) was added. Filtration provided 2-thioxo-3-(3,4,5-trimethoxy-benzyl)-thiazolidin-4-one (1.08 g, 69% yield).

D. 4-{5-[4-Oxo-2-thioxo-3-(3,4,5-trimethoxy-benzyl)-thiazolidin-5-ylidenemethyl]-furan-2-yl}-benzoic Acid Methyl Ester

To a solution of 4-(5-formyl-furan-2-yl)-benzoic acid methyl ester (108 mg, 0.5 mmol) and 2-thioxo-3-(3,4,5-trimethoxy-benzyl)-thiazolidin-4-one (157 mg, 0.5 mmol) in ethanol (10 ml) was added piperidine (1 drop). After heating at reflux for 4 hours, the mixture was concentrated under reduced pressure. The residue was purified by flash chromatography on silica gel eluting with 10% methanol-dichloromethane to give the title compound (98 mg, 37% yield).

### Example 3

### 4-{5-[4-Oxo-2-Thioxo-3-(3,4,5-trimethoxy-benzyl)-thiazolidin-5-ylidenemethyl]-furan-2-yl}-benzoic Acid

To a solution of 4-(5-formyl-furan-2-yl)-benzoic acid (43 mg, 0.2 mmol) and 2-thioxo-3-(3,4,5-trimethoxy-benzyl)-thiazolidin-4-one (63 mg, 0.2 mmol) in ethanol (10 ml) was added piperidine (1 drop). After heating at reflux for 1 hour, the mixture was cooled to room temperature acidified to pH 2 by slow addition of 0.1 M aqueous hydrochloric acid. The solid was collected by filtration to give the title compound (39 mg, 77% yield).

### Example 4

### 4-{5-[3-(3-Morpholin-4-yl-propyl)-4-oxo-2-thioxo-thiazolidin-5-ylidenemethyl]-furan-2-yl}-benzoic Acid Methyl Ester

A. 3-(3-Morpholin-4yl-propyl)-2-thioxo-thiazolidin-4-one

A suspension of bis(carboxymethyl) trithiocarbonate (498 mg, 2.2 mmol), potassium carbonate (138 mg, 1 mmol) and 4-(3-aminopropyl)morpholine (288 mg, 2 mmol) in water (20 ml) was heated at reflux for 12 hours. After treating with water (10 ml), the mixture was cooled to room temperature and extracted with 10% methanol-dichloromethane (3×50 ml). The combined extracts were dried over anhydrous magnesium sulfate and concentrated under reduced pressure. The residue was purified by flash chromatography on silica gel eluting with 10% methanol-dichloromethane to provide 3-(3-morpholin-4yl-propyl)-2-thioxo-thiazolidin-4-one (252 mg, 48% yield).

B. 4-{5-[3-(3-Morpholin-4-yl-propyl)-4-oxo-2-thioxo-thiazolidin-5-ylidenemethyl]-furan-2-yl}-benzoic Acid Methyl Ester

To a solution of 4-(5-formyl-furan-2-yl)-benzoic acid methyl ester (59 mg, 0.27 mmol) and 3-(3-morpholin-4yl-propyl)-2-thioxo-thiazolidin-4-one (72 mg, 0.27 mmol) in ethanol (10 ml) was added piperidine (1 drop). After heating at reflux for 6 hours, the mixture was concentrated under reduced pressure. The residue was purified by flash chromatography on silica gel eluting with 10% methanol-dichloromethane to give the title compound (32 mg, 25% yield).

### Example 5

### 4-[5-(4-Oxo-3-phenyl-2-thioxo-thiazolidin-5-ylidenemethyl)-furan-2-yl]-benzoic Acid

A. 3-Phenyl-2-thioxo-thiazolidin-4-one

A suspension of bis(carboxymethyl) trithiocarbonate (498 mg, 2.2 mmol), potassium carbonate (138 mg, 1 mmol) and aniline (186 mg, 2 mmol) in water (15 ml) was heated at reflux for 12 hours. After treating with water (10 ml), the mixture was cooled to room temperature and extracted with 10% methanol-dichloromethane (3×50 ml). The combined extracts were dried over anhydrous magnesium sulfate and concentrated under reduced pressure. The residue was purified by flash chromatography on silica gel eluting with ethyl acetate to provide 3-phenyl-2-thioxo-thiazolidin-4-one (225 mg, 54% yield).

B. 4-[5-(4-Oxo-3-phenyl-2-thioxo-thiazolidin-5-ylidenemethyl)-furan-2-yl]-benzoic Acid

To a solution of 4-(5-formyl-furan-2-yl)-benzoic acid (43 mg, 0.2 mmol) and 3-phenyl-2-thioxo-thiazolidin-4-one (42 mg, 0.2 mmol) in ethanol (10 ml) was added piperidine (1 drop). After heating at reflux for 30 minutes, the mixture was cooled to room temperature and treated with ethyl acetate (10 ml). The mixture was acidified to pH 2 by addition of 0.1 M hydrochloric acid. The precipitate was filtered, washed with dichloromethane (2×5 ml), and dried under vacuum to provide the title compound (40 mg, 49% yield).

### Example 6

### 4-[5-(3-Benzyl-4-oxo-2-thioxo-thiazolidin-5-ylidenemethyl)-furan-2-yl]-benzoic Acid Methyl Ester

A. 3-Benzyl-2-thioxo-thiazolidin-4-one

A suspension of bis(carboxymethyl) trithiocarbonate (2.5 g, 11 mmol), potassium carbonate (690 mg, 5 mmol) and benzylamine (1.07 g, 10 mmol) in water (15 ml) was heated at reflux for 12 hours. After treating with water (10 ml), the mixture was cooled to room temperature and extracted with 10% methanol-dichloromethane (4×50 ml). The combined extracts were washed with water (50 ml), dried over anhydrous magnesium sulfate, and concentrated under reduced pressure. The residue was purified by flash chromatography on silica gel eluting with 70% dichloromethane-hexane to provide 3-benzyl-2-thioxo-thiazolidin-4-one (960 mg, 43% yield).

B. 4-[5-(3-Benzyl-4-oxo-2-thioxo-thiazolidin-5-ylidenemethyl)-furan-2-yl]-benzoic Acid Methyl Ester

To a solution of 4-(5-formyl-furan-2-yl)-benzoic acid methyl ester (115 mg, 0.5 mmol) and 3-benzyl-2-thioxo-thiazolidin-4-one (112 mg, 0.5 mmol) in ethanol (10 ml) was added piperidine (1 drop). After heating at reflux for 30 minutes, the mixture was cooled to 0-5° C. The solid was filtered, washed with cold ethanol (2×5 ml), and dried under vacuum to give the title compound (150 mg, 69% yield).

### Example 7

### 4-[5-(2,4-Dioxo-thiazolidin-5-ylidenemethyl)-furan-2-yl]-benzoic Acid Methyl Ester

To a solution of 4-(5-formyl-furan-2-yl)-benzoic acid methyl ester (477 mg, 2.2 mmol) and 2,4-thiazolidinedione (259 mg, 2.2 mmol) in ethanol (15 ml) was added piperidine (1 drop). After heating at reflux for 4 hours, the mixture was cooled to room temperature. The solid was filtered, washed with cold ethanol (2×5 ml), and dried under the vacuum to give the title compound (534 mg, 77% yield).

### Example 8

### 4-[5-(3-Cyclohexylmethyl-2,4-Dioxo-thiazolidin-5-ylidenemethyl)-furan-2-yl]-benzoic Acid Methyl Ester

To a suspension of cesium carbonate (72 mg, 0.2 mmol) and the compound of Example 16 (66 mg, 0.2 mmol) in N,N-dimethylformamide (5 ml) was added cyclohexylmethyl bromide (35 mg, 0.2 mmol). After heating at 50-60° C. for 6 hours, the N,N-dimethylformamide was removed by evaporation under reduced pressure and the residue was partitioned between water (20 ml) and dichloromethane (50 ml). The organic layer was dried over anhydrous magnesium sulfate and concentrated under reduced pressure. Purification by flash chromatography on silica gel eluting with 90% dichloromethane-hexane gave the title compound (19 mg, 22% yield).

### Example 9

### 4-[5-(3-Cyclopropylmethyl-2,4-dioxo-thiazolidin-5-ylidenemethyl)-furan-2-yl]-benzoic Acid Methyl Ester

To a suspension of cesium carbonate (72 mg, 0.2 mmol) and the compound of Example 7 (66 mg, 0.2 mmol) in N,N-dimethylformamide (5 ml) was added cyclopropylmethyl bromide (24 mg, 0.2 mmol). After heating at 60-70° C. for 6 hours, the N,N-dimethylformamide was removed by evaporation under reduced pressure and the residue was partitioned between water (20 ml) and dichloromethane (50 ml). The organic layer was dried over anhydrous magnesium sulfate and concentrated under reduced pressure. Purification by flash chromatography on silica gel eluting with 80% dichloromethane-hexane gave the title compound (28 mg, 73% yield).

### Example 10

### 4-[4-Oxo-5-(3-phenyl-allylidene)-2-thioxo-thiazolidin-3-yl]-benzoic Acid

To a solution of 4-(4-oxo-2-thioxo-thiazolidin-3-yl)-benzoic acid, prepared according to the method described by Bailey, T. R., Young, D. C. Compounds, Compositions and Methods for Treating or Preventing Viral Infections and Associated Diseases, PCT International Application WO 00/10573 (Mar. 2, 2000) (845 mg, 3.34 mmol) and piperidine (0.33 ml, 3.34 mmol) in absolute ethanol (10 ml) was added cinnamaldehyde (0.44 ml, 3.50 mmol). After stirring at room temperature for 18 hours the solution was heated at 50° C. for 30 minutes. After cooling to room temperature, the orange solid was filtered. The solid was purified as follows. A suspension of the solid in dimethylformamide (10 ml) was treated with 3 M hydrochloric acid to pH 1. To the resulting solution, water (40 ml) was added dropwise. The solid was filtered, suspended in ethyl acetate (20 ml), again filtered, and dried under reduced pressure to afford the title compound (330 mg, 27% yield) as an orange solid.

### Example 11

### 4-{5-[4-Oxo-2-thioxo-3-(2-trifluoromethyl-benzyl)-thiazolidin-5-ylidenemethyl]-furan-2-yl}-benzoic Acid

A. 3-(2-Trifluoromethylbenzyl)-2-thioxo-thiazolidin-4-one

A suspension of bis(carboxymethyl) trithiocarbonate (498 mg, 2.2 mmol), potassium carbonate (138 mg, 1 mmol) and 2-trifluoromethylbenzyl amine (175 mg, 1 mmol) in water (20 ml) was heated under reflux for 12 hours. After cooling to room temperature, the mixture was treated with water (10 ml) and the solid was filtered to provide 3-(2-trifluoromethylbenzyl)-2-thioxo-thiazolidin-4-one (0.17 g, 61% yield).

B. 4-{5-[4-Oxo-2-thioxo-3-(2-trifluoromethyl-benzyl)-thiazolidin-5-ylidenemethyl]-furan-2-yl}-benzoic Acid

Piperidine (1 drop) was added to a solution of 4-(5-formyl-furan-2-yl)-benzoic acid (27 mg, 0.125 mmol) and 3-(2-trifluoromethylbenzyl)-2-thioxo-thiazolidin-4-one (35 mg, 0.12 mmol) in ethanol (10 ml) and the mixture was heated under reflux for 30 minutes. After cooling to 0-5° C., the mixture was acidified to pH 2.5 by addition of 0.1 M hydrochloric acid. The solid was filtered, washed with cold ethanol (2×5 ml), and dried under vacuum to give the title compound (28 mg, 47% yield).

### Example 12

### 4-{5-[3-(4-Methyl-benzyl)-4-oxo-2-thioxo-thiazolidin-5-ylidenemethyl]-furan-2-yl}-benzoic Acid

A. 3-(4-Methyl-benzyl)-2-thioxo-thiazolidin-4-one

A suspension of bis(carboxymethyl) trithiocarbonate (905 mg, 4 mmol), potassium carbonate (276 mg, 2 mmol) and 4-methylbenzyl amine (242 mg, 2 mmol) in water (20 ml) was heated under reflux for 4 hours. After cooling to room temperature, the mixture was treated with water (10 ml) and filtered to provide 3-(4-methyl-benzyl)-2-thioxo-thiazolidin-4-one (0.25 g, 53% yield).

B. 4-{5-[3-(4-Methyl-benzyl)-4-oxo-2-thioxo-thiazolidin-5-ylidenemethyl]-furan-2-yl}-benzoic Acid

Piperidine (1 drop) was added to a solution of 4-(5-formyl-furan-2-yl)-benzoic acid (43 mg, 0.2 mmol) and 3-(4-methyl-benzyl)-2-thioxo-thiazolidin-4-one (47 mg, 0.2 mmol) in ethanol (10 ml) and the mixture was heated under reflux for 2 hours. After cooling to 0-5° C., the solid was filtered, washed with cold ethanol (2×5 ml), and dried under vacuum to give the title compound (25 mg, 57% yield).

### Example 13

### 4-{5-[4-Oxo-2-thioxo-3-(3,4,5-trimethoxy-benzyl)-thiazolidin-5-ylidenemethyl]-thiophen-2-yl}-benzoic Acid Methyl Ester

A. 4-(5-Formyl-thiophene-2-yl)-benzoic Acid Methyl Ester

To a solution of 5-bromothiophene-2-carboxaldehyde (3.82 g, 20 mmol), 4-(methoxycarbonyl) phenyl boronic acid (3.60 g, 20 mmol), tris (dibenzylideneacetone) dipalladium (0) (275 mg, 0.3 mmol) and potassium fluoride (3.49 g, 60 mmol) in 1,4-doxane (100 ml) was added a solution of tri-t-butylphosphine in hexane (10 weight %, 1.45 g, 0.72 mmol). After stirring at 65-70° C. for 4 hours, the mixture was concentrated under reduced pressure and the residue was partitioned between water (100 ml) and dichloromethane (150 ml) and stirred for 10 minutes. The organic layer was separated, dried over anhydrous magnesium sulfate, and concentrated under reduced pressure. The residue was purified by flash chromatography on silica gel eluting with 50% ethyl acetate-hexane to provide 4-(5-formyl-thiophene-2-yl)-benzoic acid methyl ester (1.98 g, 40% yield).

B. 4-(5-Formyl-thiophene-2-yl)-benzoic Acid

To a solution of 4-(5-formyl-thiophene-2-yl)-benzoic acid methyl ester (369 mg, 1.5 mmol) in methanol (50 ml) and water (10 ml) was added lithium hydroxide (205 mg, 5 mmol). After stirring at room temperature for 24 hours, the solution was acidified to pH 2 by addition of 0.1 M hydrochloric acid and extracted with ethyl acetate (4×20 ml). The combined organic extracts were dried over anhydrous magnesium sulfate and concentrated under reduced pressure. The residue was purified by flash chromatography on silica gel eluting with 10% methanol-dichloromethane to provide 4-(5-formyl-thiophene-2-yl)-benzoic acid (185 mg, 53% yield).

C. 4-{5-[4-Oxo-2-thioxo-3-(3,4,5-trimethoxy-benzyl)-thiazolidin-5-ylidenemethyl]-thiophen-2-yl}-benzoic Acid Methyl Ester

Piperidine (1 drop) was added to a solution of 4-(5-formyl-thiophene-2-yl)-benzoic acid methyl ester (49 mg, 0.2 mmol) and 2-thioxo-3-(3,4,5-trimethoxy-benzyl)-thiazolidin-4-one (62.6 mg, 0.2 mmol) in ethanol (10 ml) and the mixture was heated under reflux for 30 minutes. After cooling to 0-5° C., the solid was filtered, washed with cold ethanol (2×5 ml), and dried under the vacuum to give the title compound (66 mg, 61% yield).

### Example 14

### 4-[5-(4-Oxo-3-thiophen-2-ylmethyl-2-thioxo-thiazolidin-5-ylidenemethyl)-thiophen-2-yl]-benzoic Acid

A. 3-Thiophen-2-ylmethyl-2-thioxo-thiazolidin-4-one

A suspension of bis (carboxymethyl) trithiocarbonate (905 mg, 4 mmol), potassium carbonate (276 mg, 2 mmol) and 2-(aminomethyl)thiophene (226 mg, 2 mmol) in water (20 ml) was heated under reflux for 4 hours. After cooling to room temperature, the mixture was diluted with water (10 ml) and filtered to provide 3-thiophen-2-ylmethyl-2-thioxo-thiazolidin-4-one (0.32 g, 70% yield).

B. 4-[5-(4-Oxo-3-thiophen-2-ylmethyl-2-thioxo-thiazolidin-5-ylidenemethyl)-thiophen-2-yl]-benzoic Acid

Piperidine (1 drop) was added to a solution of 4-(5-formyl-thiophene-2-yl)-benzoic acid (23.2 mg, 0.1 mmol) and 3-thiophen-2-ylmethyl-2-thioxo-thiazolidin-4-one (22.9 mg, 0.1 mmol) in ethanol (5 ml) and the mixture was heated under reflux for 60 minutes. After cooling to room temperature, the solid was filtered, washed with cold ethanol (2×5 ml), and dried under vacuum to give the title compound (25 mg, 56% yield).

### Example 15

### 4-{5-[3-(4-Methyl-benzyl)-4-oxo-2-thioxo-thiazolidin-5-ylidenemethyl]-furan-2-yl}-benzenesulfonamide

A. 4-Tributylstannylbenzenesulfinic Acid Amide

To a solution of 4-bromophenylsulfonamide (11.8 g, 50 mmol) in tetrahydrofuran (120 ml), cooled to −78° C., was added a solution of n-butyl lithium in heptane (2.7 M, 40 ml, 108 mmol) and stirred at −78° C. for 2 hours. A solution of tributyltin chloride (17.9 g, 55 mmol) in tetrahydrofuran (20 ml) was added dropwise and stirring at −78° C. was continued for 1 hour. After warming to room temperature over a period of 2 hours, saturated aqueous ammonium chloride solution (20 ml) was added and the mixture was extracted with ethyl acetate (3×45 ml). The combined organic extracts were dried over anhydrous magnesium sulfate and concentrated under reduced pressure. The residue was purified by flash chromatography on silica gel eluting with 20% ethyl acetate-hexane to provide 4-tributylstannylbenzenesulfinic acid amide (2.77 g, 12% yield).

B. 4-(5-Formyl-furan-2-yl)benzenesulfinic Acid Amide

A mixture of 4-(tributylstannyl)benzenesulfinic acid amide (2.77 g, 6.21 mmol), 5-bromofuraldehyde (1.08 g, 6.21 mmol) and bis(triphenylphosphine) palladium (II) chloride (436 mg) in ethanol (65 ml) was heated under reflux for 18 hours and under an argon atmosphere. After cooling to room temperature, the mixture was treated with ethyl ether (200 ml) and filtered through a pad of celite under suction. The filtrate was concentrated under reduced pressure. The residue was purified by flash chromatography on silica gel eluting with 20% ethyl acetate-hexane to provide 4-(5-formyl-furan-2-yl)benzenesulfinic acid amide (450 mg, 29% yield).

C. 4-{5-[3-(4-Methyl-benzyl)-4-oxo-2-thioxo-thiazolidin-5-ylidenemethyl]-furan-2-yl}-benzenesulfonamide

Piperidine (1 drop) was added to a solution of 4-(5-formyl-furan-2-yl)benzenesulfinic acid amide (50.2 mg, 0.2 mmol) and 3-(4-methyl-benzyl)-2-thioxo-thiazolidin-4-one (47.4 mg, 0.2 mmol) in ethanol (5 ml) and the mixture was heated under reflux for 30 minutes. After cooling to 0-5° C., the solid was filtered, washed with cold ethanol (2×5 ml), and dried under vacuum to give the title compound (43 mg, 91 % yield).

### Example 16

### 4-[5-(4-Oxo-3-pyridin-3-ylmethyl-2-thioxo-thiazolidin-5-ylidenemethyl)-furan-2-yl]-benzonitrile

A. 4-(5-Formyl-furan-2-yl)benzonitrile

A mixture of 4-cyanophenylboronic acid (2.94 g, 20 mmol), 5-bromofuraldehyde (3.5 g, 20 mmol), tris(dibenzylideneacetone dipalladium (0) (275 mg, 0.3 mmol), tri-t-butylphosphine (145 mg, 0.72 mmol) and potassium fluoride (3.5 g, 60 mmol) in dioxane (100 ml) was heated to 80-100° C. for 14 hours under an argon atmosphere. After cooling to room temperature, ethyl ether (200 ml) was added and the mixture was filtered through a pad of celite under suction. The filtrate was concentrated under reduced pressure. The residue was purified by flash chromatography on silica gel eluting with 50% ethyl acetate-hexane to provide 4-(5-formyl-furan-2-yl)benzonitrile (2.6 g, 66% yield).

B. 3-Pyridin-3-ylmethyl-2-thioxo-thiazolidin-4-one

A suspension of bis (carboxymethyl) trithiocarbonate (995 mg, 4.4 mmol), potassium carbonate (276 mg, 2 mmol) and 4-aminomethyl pyridine (216 mg, 2 mmol) in water (20 ml) was heated under reflux for 6 hours. After cooling to room temperature, water (10 ml) was added. The solid was filtered and purified by flash chromatography on silica gel eluting with 50% dichloromethane-hexane to provide 3-pyridin-3-ylmethyl-2-thioxo-thiazolidin-4-one (198 mg, 44% yield).

C. 4-[5-(4-Oxo-3-pyridin-3-ylmethyl-2-thioxo-thiazolidin-5-ylidenemethyl)-furan-2-yl]-benzonitrile

Piperidine (1 drop) was added to a solution of 4-(5-formyl-furan-2-yl)benzonitrile (39 mg, 0.2 mmol) and 3-pyridin-3-ylmethyl-2-thioxo-thiazolidin-4-one (44.8 mg, 0.2 mmol) in ethanol (10 ml) and the mixture was heated under reflux for 2 hours. After cooling to room temperature, the solid was filtered, washed with cold ethanol (2×5 ml), and dried under vacuum to give the title compound (70 mg, 86% yield).

### Example 17

### 4-{5-[3-(4-Dimethylamino-phenyl)-allylidene]-4-oxo-2-thioxo-thiazolidin-3-yl}-benzoic Acid

A mixture of 4-(4-oxo-2-thioxo-thiazolidin-3-yl)-benzoic acid (275 mg, 1.09 mmol), 4-(N,N-dimethylamino)cinnamaldehyde (190 mg, 1.09 mmol), and absolute ethanol (10 ml) was heated under reflux and piperidine (0.005 ml) was added. After heating under reflux for 18 hours, the mixture was cooled to room temperature and 3 M hydrochloric acid (1 ml) was added followed by water (40 ml). The solid was filtered and air dried to afford the title compound (94 mg, 21% yield) as a purple powder. ¹H NMR 300 MHz (d₆ DMSO) δ 8.09 (d, 2H) 7.6-7.49 (m, 5H) 7.33 (d, 1H) 6.86 (dd, 1H) 6.74 (d, 2H).

### Example 18

### 4-(5-Benzofuran-2-ylmethylene-4-oxo-2-thioxo-thiazolidin-3-yl)-benzoic Acid

A mixture of 4-(4-oxo-2-thioxo-thiazolidin-3-yl)-benzoic acid (185 mg, 0.73 mmol), 2-benzofuraldehyde (0.09 ml, 0.73), absolute ethanol (10 ml), dimethylsulfoxide (5 ml) and piperidine (20 ml) was heated to 95° C. for 2 hours. Dimethylsulfoxide was added until all the suspended solids had dissolved and heating at 95° C. was resumed for 18 hours. After cooling to room temperature, 3 M hydrochloric acid (0.3 ml) was added followed by water (30 ml). The solid was filtered and air dried to afford the title compound (37 mg, 13% yield) as an orange powder. ¹H NMR 300 MHz (d₆ DMSO) δ 8.12 (d, 2H) 7.99 (s, 1H) 7.81 (t, 2H) 7.68 (s, 1H) 7.59 (d, 2H) 7.51 (dt, 1H) 7.37 (t, 1H).

All above patents, patent applications and publications mentioned in this specification are incorporated by reference in their entirety to the same extent as if each individual patent, patent application or publication was specifically and individually incorporated by reference.

From the foregoing description, although specific embodiments of the invention have been described herein for purposes of illustration, one of ordinary skill in the art can easily ascertain the essential characteristics of this invention, and without departing from the spirit and scope thereof, can make various changes and modifications of the invention without undue experimentation.

We claim:

1. A method for inhibiting phospholipase D (PLD), comprising contacting PLD with an amount of a thiazolidinone or a pharmaceutically acceptable salt, composition and prodrug thereof, effective in inhibiting PLD.

2. The method of claim 1, wherein said thiazolidinone is a monocyclic thiazolidinone.

3. The method of claim 1, wherein said thiazolidinone is a bicyclic thiazolidinone.

4. The method of claim 1, wherein said thiazolidinone is a tricyclic thiazolidinone.

5. The method of claim 2, wherein said monocyclic thiazolidinone has the Formula: 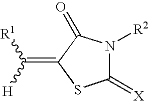
wherein R¹ is hydrogen, alkyl, alkenyl, aryl or heteroaryl; R² is hydrogen, alkyl, cycloalkyl, heteroalicyclic, aryl, arylalkyl or arylalkenyl; X is O or S; and pharmaceutically acceptable salts, compositions or prodrugs thereof.

6. The method of claim 5, wherein said heteroaryl is a substituted furan and X is S.

7. The method of claim 5, wherein R¹ is selected from H; alkyl or aryl substituted alkyl; alkyl or aryl substituted alkenyl; unsubstituted, halo, hydroxy, alkoxy, amino, alkylamino, dialkylamino, alkoxycarbonyl, aminocarbonyl, alkylmercapto or arylmercapto substituted aryl; halo, hydroxy, alkoxy, methylenedioxy, amino, alkylamino, dialkylamino, cyano, carboxy, alkoxycarbonyl, aminocarbonyl, sulfonamidyl or aminosulfonyl substituted aryl substituted furanyl, pyrrolyl or thiophenyl; halo, hydroxy, alkoxy, methylenedioxy, amino, alkylamino, dialkylamino, cyano, carboxy, alkoxycarbonyl, aminocarbonyl, aminosulfonyl or alkyl substituted aryl substituted alkyl or alkenyl; indolyl; pyridinyl; and R² is selected from H; unsubstituted or morpholino, halo, hydroxy, alkoxy, methylenedioxy, amino, alkylamino, dialkylamino, cyano, carboxy, alkoxycarbonyl, aminocarbonyl, alkylmercapto or arylmercapto substituted aryl; halo, hydroxy, alkoxy, methylenedioxy, amino, alkylamino, dialkylamino, cyano, carboxy, alkoxycarbonyl, aminocarbonyl, sulfonamidyl or aminosulfonyl pyridinyl, thiophenyl, furanyl, or pyrrolyl substituted cyclic or acyclic alkyl or alkenyl.

8. The method of claim 7, wherein R¹ is halo substituted phenyl, aryl substituted thiophenyl, or aryl substituted furanyl.

9. The method of claim 7, wherein R² is halo substituted aryl, carboxy substituted aryl, or carboxy substituted cyclic or acyclic alkyl.

10. The method according to claims 3 or 4, wherein said thiazolidinone has the Formula: 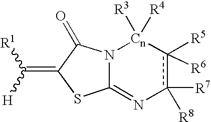
wherein the dotted line represents a double or a single bond; R¹ is alkenyl, aryl or heteroaryl; R³ and R⁴ are each independently hydrogen, alkyl or aryl; R⁵, R⁶, R⁷ and R⁸ are each independently hydrogen, alkyl, acyl or aryl; or R⁵ and R⁷, combined, form an aryl, heteroaryl, cycloalkyl or heteroalicyclic ring; and n is an integer between 0 and 2; and pharmaceutically acceptable salts, composition or prodrugs thereof;
with the proviso that:
(a) when n is 0, R³ and R⁴ are absent; and
(b) when the dotted line represents a double bond, R⁵ and R⁷ are absent.

11. The method of claim 10, wherein R¹ is heteroaryl.

12. The method of claim 11, wherein the heteroaryl is aryl substituted furanyl.

13. The method of claim 12, wherein the heteroaryl is carboxyphenyl substituted furanyl.

14. The method of claim 5, wherein said thiazolidinone is any one of compounds 1-41 of Table 1, or pharmaceutically acceptable salts, compositions and prodrugs thereof.

15. The method of claim 10, wherein said thiazolidinone is any one of compounds 42-61 of Table 2 and Table 3, or pharmaceutically acceptable salts, compositions and prodrugs thereof.

16. A method for treating cancer, comprising administering to a subject in need thereof an effective amount of a thiazolidinone, or a pharmaceutically acceptable salt, composition and prodrug thereof.

17. The method of claim 16, wherein said cancer is breast or renal cancer.

18. The method of claim 16, wherein said thiazolidinone is a monocyclic thiazolidinone.

19. The method of claim 16, wherein said thiazolidinone is a bicyclic thiazolidinone.

20. The method of claim 16, wherein said thiazolidinone is a tricyclic thiazolidinone.

21. The method of claim 18, wherein said monocyclic thiazolidinone has the Formula: 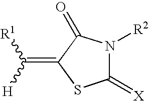
wherein R¹ is hydrogen, alkyl, alkenyl, aryl or heteroaryl; R² is hydrogen, alkyl, cycloalkyl, heteroalicyclic, aryl, arylalkyl or arylalkenyl; X is O or S; and pharmaceutically acceptable salts, compositions or prodrugs thereof.

22. The method of claim 21, wherein said heteroaryl is a substituted furan and X is S.

23. The method of claim 21, wherein R¹ is selected from H; alkyl or aryl substituted alkyl; alkyl or aryl substituted alkenyl; unsubstituted, halo, hydroxy, alkoxy, amino, alkylamino, dialkylamino, alkoxycarbonyl, aminocarbonyl, alkylmercapto or arylmercapto substituted aryl; halo, hydroxy, alkoxy, methylenedioxy, amino, alkylamino, dialkylamino, cyano, carboxy, alkoxycarbonyl, aminocarbonyl, sulfonamidyl or aminosulfonyl substituted aryl substituted furanyl, pyrrolyl or thiophenyl; halo, hydroxy, alkoxy, methylenedioxy, amino, alkylamino, dialkylamino, cyano, carboxy, alkoxycarbonyl, aminocarbonyl, aminosulfonyl or alkyl substituted aryl substituted alkyl or alkenyl; indolyl; pyridinyl; and R² is selected from H; unsubstituted or morpholino, halo, hydroxy, alkoxy, methylenedioxy, amino, alkylamino, dialkylamino, cyano, carboxy, alkoxycarbonyl, aminocarbonyl, alkylmercapto or arylmercapto substituted aryl; halo, hydroxy, alkoxy, methylenedioxy, amino, alkylamino, dialkylamino, cyano, carboxy, alkoxycarbonyl, aminocarbonyl, sulfonamidyl or aminosulfonyl pyridinyl, thiophenyl, furanyl, or pyrrolyl substituted cyclic or acyclic alkyl or alkenyl.

24. The method of claim 23, wherein R¹ is halo substituted phenyl, aryl substituted thiophenyl, or aryl substituted furanyl.

25. The method of claim 23, wherein R² is halo substituted aryl, carboxy substituted aryl, or carboxy substituted cyclic or acyclic alkyl.

26. The method according to claims 19 or 20, wherein said thiazolidinone has the Formula: 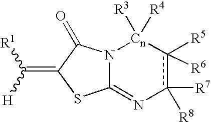
wherein the dotted line represents a double or a single bond; R¹ is alkcenyl, aryl or heteroaryl; R³ and R⁴ are each independently hydrogen, alkyl or aryl; R⁵, R⁶, R⁷ and R⁸ are each independently hydrogen, alkyl, acyl or aryl; or R⁵ and R⁷, combined, form an aryl, heteroaryl, cycloalkyl or heteroalicyclic ring; and n is an integer between 0 and 2; and pharmaceutically acceptable salts, compositions and prodrugs thereof;
with the proviso that:
(a) when n is 0, R³ and R⁴ are absent; and
(b) when the dotted line represents a double bond, R⁵ and R⁷ are absent.

27. The method of claim 26, wherein R¹ is heteroaryl.

28. The method of claim 27, wherein the heteroaryl is aryl substituted furanyl.

29. The method of claim 28, wherein the heteroaryl is carboxyphenyl substituted furanyl.

30. The method of claim 21, wherein said thiazolidinone is any one of compounds 1-41 of Table 1, or pharmaceutically acceptable salts, compositions and prodrugs thereof.

31. The method of claim 26, wherein said thiazolidinone is any one of compounds 42-61 of Table 2 and Table 3, or pharmaceutically acceptable salts, compositions and prodrugs thereof.

32. A method for treating inflammation, comprising administering to an animal in need thereof an effective amount of a thiazolidinone, or a pharmaceutically acceptable salt, composition and prodrug thereof.

33. The method of claim 32, wherein said thiazolidinone is a monocyclic thiazolidinone.

34. The method of claim 32, wherein said thiazolidinone is a bicyclic thiazolidinone.

35. The method of claim 32, wherein said thiazolidinone is a tricyclic thiazolidinone.

36. The method of claim 33, wherein said monocyclic thiazolidinone has the Formula: 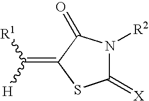
wherein R¹ is hydrogen, alkyl, alkenyl, aryl or heteroaryl; R² is hydrogen, alkyl, cycloalkyl, heteroalicyclic, aryl, arylalkyl or arylalkenyl; X is O or S; and pharmaceutically acceptable salts, compositions or prodrugs thereof.

37. The method of claim 36, wherein said heteroaryl is a substituted furan and X is S.

38. The method of claim 36, wherein R¹ is selected from H; alkyl or aryl substituted alkyl; alkyl or aryl substituted alkenyl; unsubstituted, halo, hydroxy, alkoxy, amino, alkylamino, dialkylamino, alkoxycarbonyl, aminocarbonyl, alkylmercapto or arylmercapto substituted aryl; halo, hydroxy, alkoxy, methylenedioxy, amino, alkylamino, dialkylamino, cyano, carboxy, alkoxycarbonyl, aminocarbonyl, sulfonamidyl or aminosulfonyl substituted aryl substituted furanyl, pyrrolyl or thiophenyl; halo, hydroxy, alkoxy, methylenedioxy, amino, alkylamino, dialkylamino, cyano, carboxy, alkoxycarbonyl, aminocarbonyl, aminosulfonyl or alkyl substituted aryl substituted alkyl or alkenyl; indolyl; pyridinyl; and R² is selected from H; unsubstituted or morpholino, halo, hydroxy, alkoxy, methylenedioxy, amino, alkylamino, dialkylamino, cyano, carboxy, alkoxycarbonyl, aminocarbonyl, alkylmercapto or arylmercapto substituted aryl; halo, hydroxy, alkoxy, methylenedioxy, amino, alkylamino, dialkylamino, cyano, carboxy, alkoxycarbonyl, aminocarbonyl, sulfonamidyl or aminosulfonyl pyridinyl, thiophenyl, furanyl, or pyrrolyl substituted cyclic or acyclic alkyl or alkenyl.

39. The method of claim 38, wherein R¹ is halo substituted phenyl, aryl substituted thiophenyl, or aryl substituted furanyl.

40. The method of claim 38, wherein R² is halo substituted aryl, carboxy substituted aryl, or carboxy substituted cyclic or acyclic alkyl.

41. The method according to claims 34 or 35, wherein said thiazolidinone has the Formula: 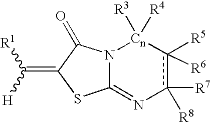
wherein the dotted line represents a double or a single bond; R¹ is alkenyl, aryl or heteroaryl; R³ and R⁴ are each independently hydrogen, alkyl or aryl; R⁵, R⁶, R⁷ and R⁸ are each independently hydrogen, alkyl, acyl or aryl; or R⁵ and R⁷, combined, form an aryl, heteroaryl, cycloalkyl or heteroalicyclic ring; and n is an integer between 0 and 2; and pharmaceutically acceptable salts, compositions and prodrugs thereof;
with the proviso that:
(a) when n is 0, R³ and R⁴ are absent; and
(b) when the dotted line represents a double bond, R⁵ and R⁷ are absent.

42. The method of claim 41, wherein R¹ is heteroaryl.

43. The method of claim 42, wherein the heteroaryl is aryl substituted furanyl.

44. The method of claim 43, wherein the heteroaryl is carboxyphenyl substituted furanyl.

45. The method of claim 36, wherein said thiazolidinone is any one of compounds 1-41 of Table 1, or pharmaceutically acceptable salts, compositions and prodrugs thereof.

46. The method of claim 41, wherein said thiazolidinone is any one of compounds 42-61 of Table 2 and Table 3, or pharmaceutically acceptable salts, compositions and prodrugs thereof.


SEQUENCE LISTING (14 sequences)

SEQ ID NO: 1 - DNA, 24 bp, Artificial Sequence
  Synthetic oligonucleotide based on putative coding sequence of the human cDNA clone genbank #204986.
gtccatgctaatgtacagttgctc

SEQ ID NO: 2 - DNA, 21 bp, Artificial Sequence
  Primer corresponding to the vector sequence just downstream of the cDNA cloning region of plasmid pCMV.SPORT
ctagcttataatacgactcac

SEQ ID NO: 3 - DNA, 24 bp, Artificial Sequence
  Primer complementary to the region corresponding to aa 804-810 of hPLDl.
ttccctgtgagctttcaggatcct

SEQ ID NO: 4 - DNA, 20 bp, Artificial Sequence
  Primer corresponding to the region near the Nhe I site in the 5′-untranslated region of hPLDl.
cgccaacgcgaggtgctagc

SEQ ID NO: 5 - DNA, 29 bp, Artificial Sequence
  Primer used to make the CNN enhancer.
ggctctagatattaatagtaatcaattac

SEQ ID NO: 6 - DNA, 26 bp, Artificial Sequence
  Primer used to make the CNN enhancer.
cctcacgcatgcaccatggtaatagc

SEQ ID NO: 7 - DNA, 24 bp, Artificial Sequence
  Primer used to make the EF-1alpha promoter and intron.
ggtgcatgcgtgaggctccggtgc

SEQ ID NO: 8 - DNA, 28 bp, Artificial Sequence
  Primer used to make the EF-1alpha promoter and intron.
gtagttttcacggtacctgaaatggaag

SEQ ID NO: 9 - DNA, 30 bp, Artificial Sequence
  Primer used to make the Baculovirus expression vectors.
gcatgtcgacctcactatagggcgaattgg

SEQ ID NO: 10 - DNA, 30 bp, Artificial Sequence
  Primer used to make the Baculovirus expression vectors.
gcattctagagctggagctccaccgcggtg

SEQ ID NO: 11 - DNA, 32 bp, Artificial Sequence
  Primer used to make the hPLD2 Baculovirus expression vectors.
gcgcggaattcggtaccaggtatgacggcgac

SEQ ID NO: 12 - DNA, 42 bp, Artificial Sequence
  Primer used to make the hPLD2 Baculovirus expression vectors.
gacaagcttgcggccgcttaattaaagatctttttttttttt

SEQ ID NO: 13 - DNA, 33 bp, Artificial Sequence
  Primer used to amplify PLD2.2 cDNA from pFBPLD2.2.
gcctacgtcgacatgacggcgacccctgagagc

SEQ ID NO: 14 - DNA, 30 bp, Artificial Sequence
  Primer used to amplify PLD2.2 cDNA from pFBPLD2.2.
caggctctagactatgtccacacttctagg